(19) United States
(12) Patent Application Publication
(10) Pub. No.: US 2021/0145834 A1
(43) Pub. Date: May 20, 2021

(54) COMBINATION OF POZIOTINIB WITH CYTOTOXIC AGENT AND/OR OTHER MOLECULARLY TARGETED AGENT AND USE THEREOF

(71) Applicant: Spectrum Pharmaceuticals, Inc., Irvine, CA (US)

(72) Inventor: Guru Reddy, Irvine, CA (US)

(21) Appl. No.: 17/250,258

(22) PCT Filed: Jun. 25, 2019
(86) PCT No.: PCT/US19/38971

§ 371 Date: Dec. 22, 2020

Related U.S. Application Data
(60) Provisional application No. 62/689,530, filed on Jun. 25, 2018.

(51) Int. Cl. A61K 31/517; C07K 16/28; A61K 31/337; A61K 31/475; A61K 33/243; A61K 31/513; C07K 16/32; A61K 45/06; A61P 35/00

(52) U.S. Cl. CPC A61K 31/517; C07K 16/2863; A61K 31/337; A61K 31/475; A61P 35/00; A61K 31/513; C07K 16/32; A61K 45/06; A61K 33/243

(57) ABSTRACT

Provided are a combination of poziotinib and at least one agent selected from the group consisting of cytotoxic agents and molecularly targeted agents; and a use of the combination.

### CROSS-REFERENCE TO RELATED APPLICATIONS

This application claims the benefit of U.S. Provisional Application No. 62/689,530, filed on Jun. 25, 2018, the entire disclosures of which are incorporated herein by reference in its entirety.

### FIELD OF THE DISCLOSURE

One or more embodiments relate to a combination drug including poziotinib with a cytotoxic agent and/or a molecularly targeted agent and a use of the drug.

### BRIEF DESCRIPTION OF RELATED TECHNOLOGY

Poziotinib is a low-molecular weight compound that selectively and irreversibly inhibits the epidermal growth factor receptor (EGFR) family, including Her1, Her2, and Her4. Poziotinib is also a pan-Her inhibitor that is highly effective at inhibiting EGFR and Her2 activation and resistance mutants. The activity of poziotinib is disclosed in U.S. Pat. No. 8,188,102B and US 2013/0071452A1, which are hereby incorporated by reference. In U.S. Pat. No. 8,188,102B, a compound of the formula 1 is a compound of Example 36.

An EGFR is the first known growth factor receptor among protein tyrosine kinases which is a protein composed of a receptor part and a tyrosine kinase part, and it transmits signals outside a cell into the cell through the cell membrane. The EGFR tyrosine kinase family has four subtype receptors of EGFR/ErbB1, Her2/ErbB2, Her3/ErbB3, and Her4/ErbB4 (hereinafter also referred to as “Her1, Her2, Her3 and Her4”, respectively). All of which can form signaling complexes as homodimers or heterodimers including two different members of the family. The EGFR plays an essential role in normal cell regulation through intracellular signal transduction, but when the signals transmitted via the receptor cannot be controlled due to overexpression or activation mutants, the EGFR activates the cell signaling system to induce growth or differentiation of tumor cells, neovascularization, metastasis, and resistance expression. (Wells A. Int J Biochem Cell Biol., 1999, 31, 637 and Nancy E. Hynes and Heidi A. Lane, Nature Reviews Cancer 5, 341, 2005). It has been reported that EGFR is abnormally overexpressed or mutated frequently in most solid cancer cells, and this is associated with poor prognosis. Therefore, studies for developing anticancer agents targeting EGFR have been actively pursued with anticipation that the anticancer effect will be excellent if the signal transmission of cancer cells through the EGFR is blocked.

EGFR-targeted anticancer agents are classified as monoclonal antibody drugs that target an extracellular domain of the receptor and as low-molecular weight drugs that target an intracellular tyrosine kinase. Examples of the EGFR-targeting antibody drugs include trastuzumab, which is a Her2-specific humanized monoclonal antibody, and Cetuximab, which is a Her1-specific chimeric (mouse/human) monoclonal antibody. Trastuzumab and Cetuximab are used as therapeutic agents alone or in combination therapy for Her2 positive advanced breast cancer and gastric cancer and for Her1 positive metastatic colorectal cancer and head and neck cancer, respectively. Examples of the EGFR-targeting low-molecular weight drugs include gefitinib and erlotinib which are selective inhibitors of EGFR/ErbB1, and lapatinib, which is an inhibitor of both EGFR and Her2. Gefitinib, and erlotinib, are used as therapeutic agents for lung cancer and lapatinib is used for Her2 positive advanced breast cancer, and clinical trials are underway to expand the indications for other solid cancer treatments.

In recent years, however, resistance expression in EGFR target therapy has been reported to decrease response time of the drug used. It has been reported that non-small-cell lung carcinoma (NSCLC) patients with EGFR activating mutations treated with gefitinib or erlotinib are resistant to the drug after about 8 to 16 months, and about 60% of the patients are resistant due to the EGFR T790M mutation (Helena A. Yu et al., Clin. Cancer Res. 19(8), 2240, 2013). In addition, in cases of Her2 positive metastatic breast cancer patients treated with the antibody drug, trastuzumab, 66% to 88% of the patients are known to exhibit de novo resistance or acquired resistance due to various mechanisms (Alice Chung et al., Clin. Breast Cancer 13(4), 223, 2013).

In this regard, the development of an EGFR targeting therapeutic agent has a limitation in that the efficacy cannot be maintained for a long period of time due to generation of primary and secondary resistance, despite the fact that the EGFR targeting. therapeutic agent has considerable effect on the treatment of solid cancers with EGFR/Her2 overexpression or mutation. Therefore, there is an urgent need for an effective therapeutic treatment that can enhance the efficacy and overcome resistance in the treatment of solid cancers with EGFR/Her2 overexpression or mutation.

### SUMMARY OF THE INVENTION

The present invention addresses this need. According to one embodiment, provided is a combination drug for treating a neoplasm associated with overexpression or amplification of at least one gene of HER1, HER2, and HER4 or a mutant of HER1, HER2, or HER4 in a subject, the combination drug including a combination of poziotinib and at least one selected from the group consisting of a cytotoxic agent and a molecularly targeted agent, as an active ingredient.

According to another embodiment, provided is a use of the combination drug in the preparation of a medicament or medicaments for treating a neoplasm associated with overexpression or amplification of at least one gene of HER1, HER2, and HER4 or a mutant of HER1, HER2, or HER4 in a subject.

According to another embodiment, provided is a method of treating a neoplasm associated with overexpression or amplification of HER1, HER2, or HER4 or a mutant of HER1, HER2, or HER4 in a subject, the method including administrating a combination of active ingredients including therapeutically effective amounts of poziotinib and at least one selected from the group consisting of a cytotoxic agent and a molecularly targeted agent.

According to another embodiment, provided is a kit for treating a neoplasm associated with overexpression or amplification of HER1, HER2, or HER4 or a mutant of HER1, HER2, or HER4 in a subject, the kit including a first part and a second part, wherein the first part includes poziotinib, and the second part includes at least one active ingredient selected from the group consisting of a cytotoxic agent and a molecularly targeted agent. The kit can also further comprise a package insert comprising instructions for treating a neoplasm associated with overexpression or amplification of HER1, HER2, or HER4 or a mutant thereof in a subject.

### BRIEF DESCRIPTION OF THE DRAWINGS

These and/or other aspects will become apparent and more readily appreciated from the following description of the embodiments, taken in conjunction with the accompanying drawings in which:

FIGS. 1A to 1D show examples of the synergistic effect in BT-474 cells, a breast cancer cell line, that are Her2-overexpressed and trastuzumab sensitive. FIG. 1A shows dose response curves of 10 μM of cisplatin and poziotinib within a concentration range of 0.25 nM to 2 nM, alone and in combination. FIG. 1B shows dose response curves of paclitaxel and poziotinib within a concentration range of 0.25 nM to 2 nM, alone and in combination at a ratio of paclitaxel:poziotinib=1:1. FIG. 10 shows dose response curves of 5 μM of 5-FU and poziotinib within a concentration range of 0.25 nM to 2 nM, alone and in combination. FIG. 1D shows dose response curves of trastuzumab within a concentration range of 5 nM to 100 nM and poziotinib within a concentration range of 0.05 nM to 1 nM, alone and in combination at a ratio of trastuzumab:poziotinib=100:1;

FIGS. 2A to 20 show examples of the synergistic effect in MDA-MB-453 cells, a breast cancer cell line, that are HER2-overexpressed, trastuzumab resistance, and with PIK3CA mutation. FIG. 2A shows dose response curves of paclitaxel and poziotinib within a concentration range of 0.3 nM to 2.5 nM, alone and in combination at a ratio of paclitaxel:poziotinib=1:1. FIG. 2B shows dose response curves of 5-FU within a concentration range of 60 nM to 500 nM and poziotinib within a concentration range of 0.3 nM to 2.5 nM, alone and or in combination at a ratio of 5-FU:poziotinib=200:1. FIG. 2C shows dose response curves of trastuzumab within a concentration range of 0.5 nM to 25 nM and poziotinib within a concentration range of 0.05 nM and 2.5 nM, alone and in combination at a ratio of trastuzumab:poziotinib=10:1;

FIG. 3 shows an example of the synergistic effect in MCF-7 cells, an estrogen receptor-positive breast cancer cell line, in which Her1 and Her2 are not overexpressed. FIG. 3 shows dose response curves of 2 nM of vinorelbine and poziotinib within a concentration range of 0.1 μM to 10 μM, alone and in combination;

FIG. 4 shows an example of the synergistic effect in MDA-MB-361 cells, a breast cancer cell line, that are Her2-overexpressed, trastuzumab resistance, and with PIK3CA mutation. FIG. 4 shows dose response curves of 1 nM of vinorelbine and poziotinib within a concentration range of 1 μM to 10 μM, alone and in combination;

FIG. 5 shows an example of the synergistic effect in TE-4 cells, an esophageal cancer cell line, that are Her2-overexpressed. FIG. 5 shows dose response curves of 5-FU within a concentration range of 10 nM to 50,000 nM and 0.5 nM of poziotinib, alone and in combination;

FIGS. 6A and 6B show examples of the synergistic effect in HCC827 cells, an NSCLC cell line, that are with Her1 L858R mutation. FIG. 6A shows dose response curves of paclitaxel within a concentration range of 0.5 nM to 20 nM and poziotinib within a concentration range of 0.05 nM to 2 nM, alone and in combination at a ratio of paclitaxel:poziotinib=10:1. FIG. 6B shows dose response curves of cisplatin within a concentration range of 0.1 nM to 4 μM and poziotinib within a concentration range of 0.05 nM to 2 nM, alone and or in combination at a ratio of cisplatin:poziototinib=2,000:1; and

FIGS. 7A and 7B show examples of the synergistic effect in H1975 cells, an NSCLC cell line carrying Her1 L858R/T790M mutation. FIG. 7A shows dose response curves of paclitaxel within a concentration range of 1.5 nM to 50 nM and poziotinib within a concentration range of 0.15 nM to 5 nM, alone and in combination at a ratio of paclitaxel:poziotinib=10:1. FIG. 7B shows dose response curves of cetuximab within a concentration range of 80 nM to 5,000 nM and poziotinib within a concentration range of 0.4 nM to 25 nM, alone and or in combination at a ratio of cetuximab:poziototinib=200:1.

FIGS. 8A and 8B display the effect of Poziotnib and Pemetrexed or Letrozole combination on Calu3 cell lines.

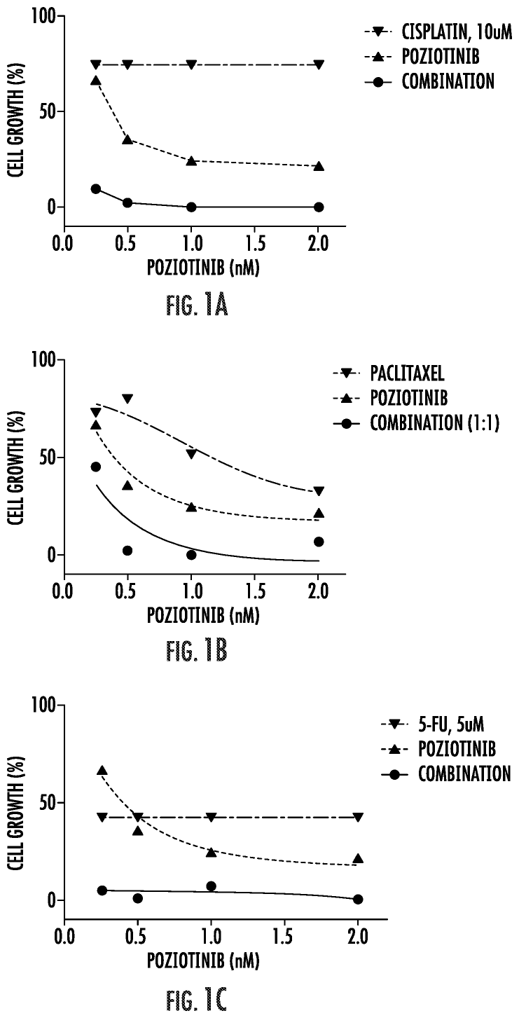

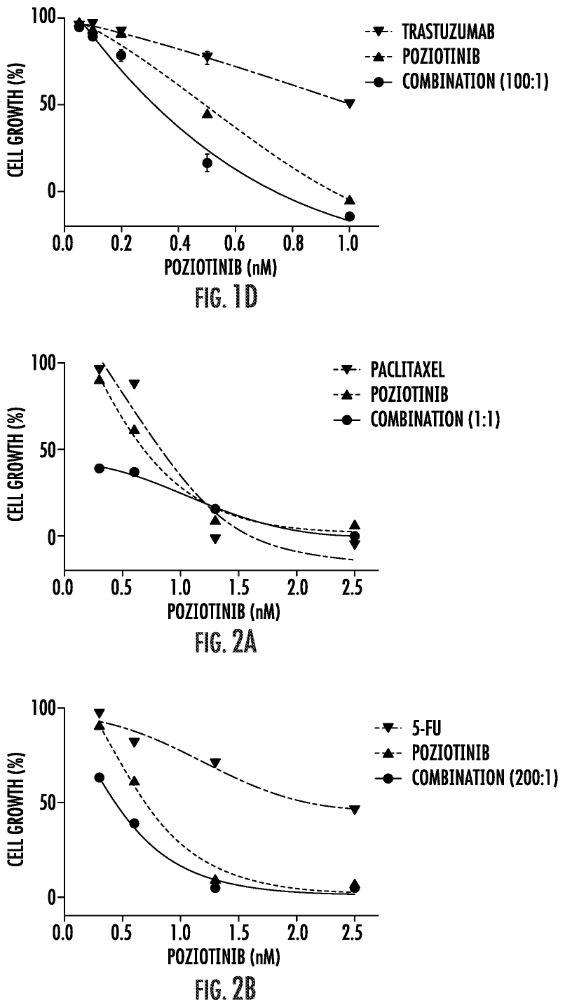

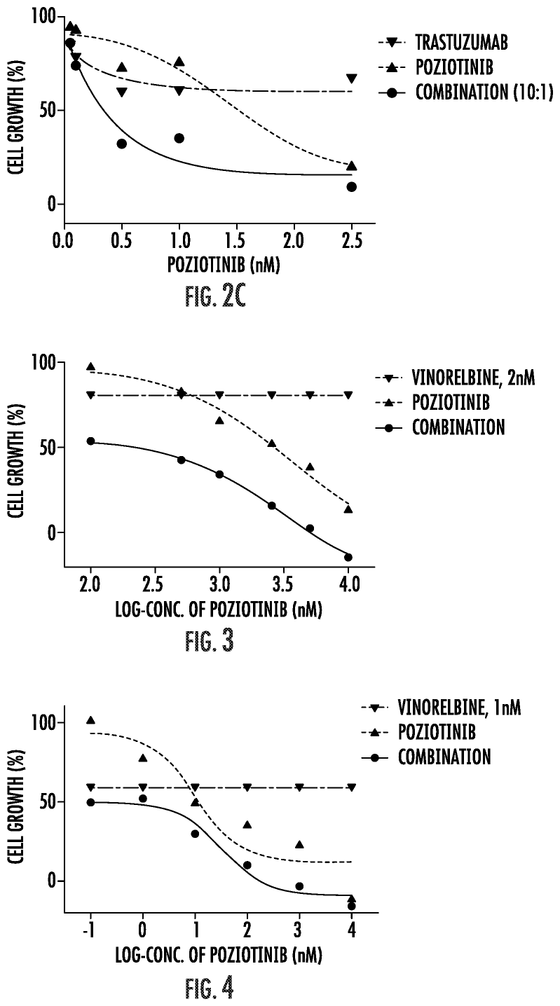

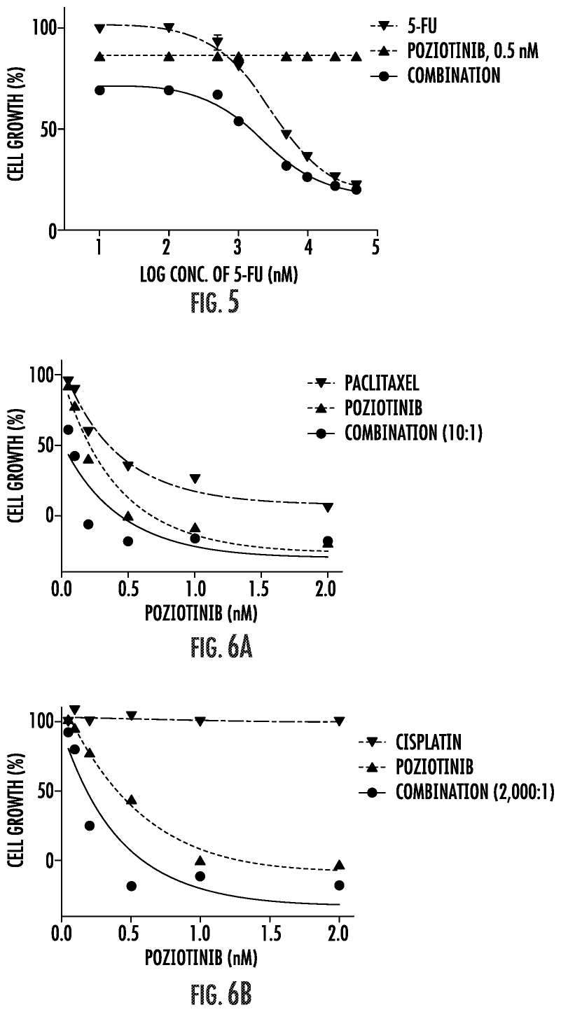

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

According to an embodiment, provided is a combination drug for treating a neoplasm. In a more specific embodiment, the neoplasm is associated with overexpression or amplification of at least one gene of HER1, HER2, and HER4 or a mutant of HER1, HER2, or HER4 in a subject, the combination drug including a combination of poziotinib and at least one cytotoxic agent and/or at least one molecularly targeted agent, as the active ingredient.

Poziotinib, i.e., 1-[4-[4-(3,4-dichloro-2-fluoroanilino)-7-methoxyquinazolin-6-yl]oxypiperidin-1-yl]prop-2-en-1-one, or pharmaceutically acceptable hydrates and/or salts thereof have a structure represented by the formula 1.

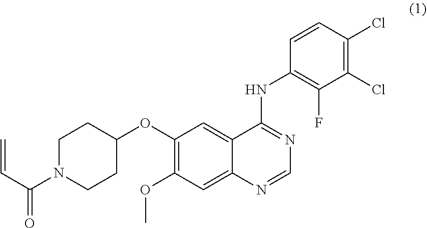 [SMILES: C=CC(=O)N1CCC(Oc2cc3c(Nc4ccc(Cl)c(Cl)c4F)ncnc3cc2OC)CC1]

The pharmaceutically acceptable salt may be an inorganic acid salt, an organic acid salt, or a metal salt. The inorganic acid salt may be a salt of hydrochloric acid, phosphoric acid, sulfuric acid, or disulfuric acid. The organic acid salt may be a salt of malic acid, maleic acid, citric acid, fumaric acid, besylic acid, camsylic acid, or edisylic acid. The metal salt may be a calcium salt, sodium salt, magnesium salt, strontium salt, or potassium salt. In one embodiment, Poziotinib may be a hydrochloride in the form of a tablet. Poziotinib may be administered in an amount of 0.1 mg to 50 mg.

Poziotinib is a low-molecular weight compound that selectively and irreversibly inhibits the epidermal growth factor receptor (EGFR) family, including Her1, Her2, and Her4. Poziotinib is also a pan-Her inhibitor that is highly effective at inhibiting EGFR and Her2 activation and resistance mutants. The activity of poziotinib is disclosed in U.S. Pat. No. 8,188,102B and US 2013/0071452A1, which are hereby incorporated by reference in their entirety. In U.S. Pat. No. 8,188,102B, a compound of the formula 1 is a compound of Example 36. Poziotinib inhibits the growth of tumor cells in various carcinomas with Her1 or Her2 overexpression or activated mutants in vitro and effectively inhibits the growth of lung cancer cells resistant to gefitinib or erlotinib. Also, it effectively blocked tumor growth in a hetero-transplantation animal model that is an animal body transplanted with such a tumor cell. In addition, poziotinib has a broad and excellent inhibitory effect on EGFR and its mutation, and the therapeutic domain can be broad and more effective, including areas resistant to other known EGFR-targeted antibody drugs and low-molecular weight drugs. Combination therapies with other drugs on this basis may also improve the effect of resistance to various solid cancers and may improve the response rate and extend the survival time compared to the effects produced by using a conventional therapeutic agent.

A cytotoxic agent refers to an agent that has a cytotoxic effect on a cell. A cytotoxic effect refers to the depletion, elimination and/or the killing of target cells (i.e., tumor cells). The cytotoxic agent may be at least one selected from the group consisting of an antimetabolite, a mitotic inhibitor, an alkylating agent, a platinum-based antineoplastic, an antibody based EGFR inhibitor, an antibody based HER2/3 inhibitor, an angiogenesis inhibitor, a mTOR inhibitor, a CDK4 and CDK6 inhibitor or an aromatase inhibitor. The combination may include at least two cytotoxic agents. For example, the combination may include at least 2, at least 3, or at least 4 selected from the group consisting of an antimetabolite, a mitotic inhibitor, an alkylating agent, an angiogenesis inhibitor and a platinum-based antineoplastic drug, or all of them.

The antimetabolite may be a drug that inhibits DNA synthesis in cells by suppressing formation of purines or pyrimidines, which are bases of a nucleotide. In one embodiment, the antimetabolite may be selected from the group consisting of Capecitabine, 5-Fluorouracil, Gemcitabine, Pemetrexed, Methotrexate, 6-Mercaptopurine, Cladribine, Cytarabine, Doxifludine, Floxuridine, Fludarabine, Hydroxycarbamide, decarbazine, hydroxyurea, and asparaginase. In a more specific embodiment, the antimetabolite is a base analog, with the term base analogs herein including nucleotide and nucleoside analogs in addition to purine base analogs such as 5-fluorouracil.

The mitotic inhibitor may be a microtubule-destabilizing agent, a microtubule-stabilizing agent, or a combination thereof. The mitotic inhibitor may be selected from taxanes, vinca alkaloids, epothilone, or a combination thereof. In a specific embodiment, the mitotic inhibitor is a taxane, for example including but not limited to, paclitaxel, docetaxel and cabaitaxel. In another specific embodiment, the mitotic inhibitor is a vinca alkaloid or its derivative, for example including but not limited to, vinblastine, vincristine, vinflunine, vinorelbine, vincaminol, vinburnine, vineridine and vindesine.

The mitotic inhibitor may be selected from BT-062, HMN-214, eribulin mesylate, vindesine, EC-1069, EC-1456, EC-531, vintafolide, 2-methoxyestradiol, GTx-230, trastuzumab emtansine (T-DM1), crolibulin, D1302A-maytansinoid conjugates IMGN-529, lorvotuzumab mertansine, SAR-3419, SAR-566658, IMP-03138, topotecan/vincristine combinations, BPH-8, fosbretabulin tromethamine, estramustine phosphate sodium, vincristine, vinflunine, vinorelbine, RX-21101, cabazitaxel, STA-9584, vinblastine, epothilone A, patupilone, ixabepilone, Epothilone D, paclitaxel, docetaxel, DJ-927, discodermolide, eleutherobin, and pharmaceutically acceptable salts thereof or combinations thereof.

As used herein, an “alkylating agent” is a substance that adds one or more alkyl groups (CₙHₘ, where n and m are integers) to a nucleic acid. In the present invention, an alkylating agent is selected from the group consisting of nitrogen mustards, nitrosoureas, alkyl sulfonates, triazines, ethylenimines, and combinations thereof. Non-limiting examples of nitrogen mustards include mechlorethamine, chlorambucil, cyclophosphamide, bendamustine, ifosfamide, melphalan, melphalan flufenamide, and pharmaceutically acceptable salts thereof. Non-limiting examples of nitrosoureas include streptozocin, carmustine, lomustine, and pharmaceutically acceptable salts thereof. Non-limiting examples of alkyl sulfonates include busulfan and pharmaceutically acceptable salts thereof. Non-limiting examples of triazines include dacarbazine, temozolomide, and pharmaceutically acceptable salts thereof. Non-limiting examples of ethylenimines include thiotepa, altretamine, and pharmaceutically acceptable salts thereof. Other alkylating agents include ProLindac™, Ac-225 BC-8, ALF-2111, trofosfamide, MDX-1203, thioureidobutyronitrile, mitobronitol, mitolactol, nimustine, glufosfamide, HuMax-TAC and PBD ADC combinations, BP-C1, treosulfan, nifurtimox, improsulfan tosilate, ranimustine, ND-01, HH-1, 22P1G cells and ifosfamide combinations, estramustine phosphate, prednimustine, lurbinectedin, trabectedin, altreatamine, SGN-CD33A, fotemustine, nedaplatin, heptaplatin, apaziquone, SG-2000, TLK-58747, laromustine, procarbazine, and pharmaceutically acceptable salts thereof.

The angiogenesis inhibitors are substances that inhibits the growth of new blood vessels (angiogenesis). Some angiogenesis inhibitors are endogenous and a normal part of the body's control and others are obtained exogenously through pharmaceutical drugs or diet. In at least one embodiment, the angiogenesis inhibitors include bevcizumab, sunitinib, sorafenib or pazopatinib.

The platinum-based antineoplastic drug may be selected from the group consisting of Cisplatin, Carboplatin, Dicycloplatin, Eptaplatin, Lobaplatin, Miriplatin, Nedaplatin, Oxaliplatin, Picoplatin, and Satraplatin.

As used herein, a “molecularly targeted agent” is a substance that interferes with the function of a single molecule or group of molecules, preferably those that are involved in tumor growth and progression, when administered to a subject. Non-limiting examples of molecularly targeted agent of the present invention include signal transduction inhibitors, modulators of gene expression and other cellular functions, immune system modulators, antibody-drug conjugates (ADCs), and combinations thereof.

The molecularly targeted agent may be selected from epidermal growth factor receptor family inhibitors (EGFRi), mammalian target of rapamycin (mTOR) inhibitors, immune checkpoint inhibitors, anaplastic lymphoma kinase (ALK) inhibitors, B-cell lymphoma-2 (BCL-2) inhibitors, B-Raf inhibitors, cyclin-dependent kinase inhibitors (CDKi), such as the CDK4/CDK6 inhibitor, palbociclib, ERK inhibitors, histone deacetylase inhibitors (HDACi), heat shock protein-90 inhibitors (HSP90i), Janus kinase inhibitors, mitogen activated protein kinase (MAPK) inhibitors, MEK inhibitors, such as the MEK1/MEK2 inhibitor trametinib, poly ADP ribose polymerase (PARP) inhibitors, phosphoinositide 3-kinase inhibitors (PI3Ki), Ras inhibitors, sodium-glucose linked transporter (SG LT) inhibitors and combinations thereof.

Suitable sodium-glucose linked transporter (SGLT) inhibitors, also known as sodium-dependent glucose cotransporter inhibitors, include inhibitors of sodium/glucose cotransporter 1 (SGLT1).

The molecularly targeted agent may be selected from ado-trastuzumab emtansine (T-DM1), alemtuzumab, cetuximab, ipilimumab, ofatumumab, panitumumab, pertuzumab, rituximab, tositumomab, 1311-tositumomab, trastuzumab, brentuximab vedotin, denileukin diftitox, ibritumomab tiuxetan, axitinib, bortezomib, bosutinib, cabozantinib, crizotinib, carfilzomib, dasatinib, erlotinib, gefitinib, imatinib mesylate, lapatinib, nilotinib, pazopanib, ponatinib, regorafenib, ruxolitinib, sorafenib, sunitinib, tofacitinib, vandetanib, vemurafenib, alitretinoin, bexarotene, everolimus, romidepsin, temsirolimus, tretinoin, vorinostat, and pharmaceutically acceptable salts thereof or combinations thereof. The molecularly targeted agent may include an antibody or an antibody moiety or an antibody-drug conjugate.

The EGFR inhibitors may be selected from erlotinib, gefitinib, lapatinib, canetinib, pelitinib, neratinib, (R,E)-N-(7-chloro-1-(1-(4-(dimethylamino)but-2-enoyl)azepan-3-yl)-1H-benzo[d]imidazol-2-yl)-2-methylisonicotinamide, Trastuzumab, Margetuximab, panitumumab, matuzumab, necitumumab, pertuzumab, nimotuzumab, zalutumumab, cetuximab, icotinib, afatinib, and pharmaceutically acceptable salt thereof. In one embodiment the EGFR inhibitor may be an antibody based EGFR inhibitor such as cetuximab and in another embodiment, it is necitumumab and yet in another embodiment it is pantitumumab. The molecularly targeted agent may be an anti-EGFR family antibody or a complex including the anti-EGFR family antibody. The anti-EGFR family antibody may be an anti-HER1 antibody, an anti-HER2 antibody, or an anti-HER4 antibody.

The neoplasm associated with overexpression or amplification of at least one gene of HER1, HER2, and HER4 or a mutant thereof may be an abnormal growth of tissue, which if it forms a mass, is commonly referred to as a tumor having overexpression of at least one of HER1, HER2, HER4 and mutant thereof or amplification of at least one gene coding of HER1, HER2, HER4 or mutant thereof. In one embodiment, the mutant can be present in any of exon 18, 19, 20 and 21 or any combinations thereof. For example, the mutant may be HER1 having exon 19 deletion, T790M substitution, L828R substitution, or combination thereof. In another embodiment, the mutation may be at HER2 exon 20, such as exon 20 insertion mutations. In another embodiment, there may be one or more mutations in exon 19 or 20 or both. For example, the one or more EGFR exon 20 mutations comprise one or more point mutations, insertions, and/or deletions of 3-18 nucleotides between amino acids 763-778.

In other embodiments, the subject may have been determined to have 2, 3, or 4 EGFR exon 20 mutations at one or more residues selected from the group consisting of A763, A767, S768, V769, D770, N771, P772, and H773. In another embodiment, the subject may be determined to not have an EGFR mutation at residue C797. In some embodiments, the one or more EGFR mutations include substitution and/or deletion at the A763, A767, S768, V769, D770, N771, P772, and H773 in exon 20. In some embodiments, the one or more exon 20 mutations are selected from the group consisting of A763insFQEA, A767insASV, S768dupSVD, V769insASV, D770insSVD, D770insNPG, H773insNPH, N771del insGY, N771del insFH, and N771dupNPH.

In certain embodiments, the genomic sample from the subject may from saliva, blood, urine, normal tissue, or tumor tissue. In particular aspects, the presence of an EGFR exon 20 mutation is determined by nucleic acid sequencing (e.g., DNA sequencing of tumor tissue or circulating free DNA from plasma) or PCR analyses.

Other embodiments provide methods of treating cancer in a patient comprising administering an effective amount of poziotinib and a secondary cytotoxic agent to the subject, wherein the subject has been determined to have one or more exon 19 or exon 20 mutations.

As used herein, “overexpression” indicates that the protein is expressed at a higher level than normal cells. The expression level can be measured using immunohistochemistry, fluorescence in situ hybridization (FISH), or chromogenic in situ hybridization (CISH). The neoplasm may be lung cancer including non-small cell lung cancer, breast cancer, stomach cancer, colon cancer, pancreatic cancer, prostate cancer, myeloma, head and neck cancer, ovarian cancer, esophageal cancer, or metastatic cell carcinoma.

The subject may be a mammal. The mammal may be a human.

The administration of a therapeutically effective amount of the combinations of the invention are advantageous over the individual component compounds in that the combinations will provide one or more of the following improved properties when compared to the individual administration of a therapeutically effective amount of a component compound: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, vi) an increase in the bioavailability of one or both of the component compounds, or vii) an increase in apoptosis over the individual component compounds.

The compounds of the invention may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood herein that each of the terms “poziotinib”, “cytotoxic agent” and “molecularly targeted agent” encompasses all closely related forms of the compound referred to as may suit the case. For instance, by each of the terms “poziotinib”, “cytotoxic agent” and “molecularly targeted agent” herein, all tautomers and mixtures of tautomers are included within the scope as well as the pharmaceutically acceptable solvates and/or salts thereof. This principle for the encompassed scope applies not only to the very terms “cytotoxic agent” and “molecularly targeted agent” but to any specific member of drug belonging to the group defined by these two terms. For example, the term “cisplatin” used herein encompasses all its tautomers and mixtures of tautomers as well as pharmaceutically acceptable solvates and/or salts thereof just as same as it would be so for the term “cytotoxic agent” when in combination with poziotinib.

The compounds of the invention may form a solvate which is understood to be a complex of variable stoichiometry formed by a solute (in this invention, the solute can be poziotinib or a salt thereof with a cytotoxic agent or a salt thereof and/or a molecularly targeted agent or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Suitably the solvent used is water.

The pharmaceutically acceptable salts of the compounds of the invention are readily prepared by those of skill in the art.

In one embodiment the at least one cytotoxic agent is selected from the group consisting of taxanes, base analogs, platinum-based and vinca alkaloids antineoplastics, and aromatase inhibitors. In at least one embodiment, the aromatase inhibitors include letrozole and anastrozole.

In another embodiment the at least one molecularly targeted agent is selected from the group consisting of epidermal growth factor receptor (EGFR) family inhibitors and mammalian target of rapamycin (mTOR) inhibitors such as everolimus, temsirolimus and sirolumus.

In a specific embodiment, the combination comprises poziotinib and an anti-EGFR family antibody. In another specific embodiment, the combination comprises poziotinib and a taxane. In still another specific embodiment, the combination comprises poziotinib and a base analog. In yet another specific embodiment, the combination comprises poziotinib and a platinum-based antineoplastic drug. In still yet another specific embodiment, the combination comprises poziotinib and a vinca alkaloid.

In one embodiment, the combination may include poziotinib and an anti-EGFR family antibody. The anti-EGFR family antibody may be trastuzumab, cetuximab, margetuximab, matuzumab, panitumumab, necitumumab, or pertuzumab. An example of the combination may be poziotinib and trastuzumab; or poziotinib and cetuximab. Poziotinib may be the hydrochloride salt form. The combination may further include a cytotoxic agent. The cytotoxic agent may be a mitotic inhibitor. The mitotic inhibitor may be taxane, vinca alkaloid, epothilone, or a combination thereof. The vinca alkaloid may be at least one drug selected from the group consisting of vinblastine, vincristine, vindesine and vinorelbine. An example of the combination may include poziotinib and trastuzumab and vinorelbine. The vinorelbine may be in the form of an injection. The taxane may be paclitaxel or docetaxel. An example of the combination may include poziotinib and cetuximab and paclitaxel. The paclitaxel may be in the form of an injection.

Poziotinib may be administered in an amount of 0.1 mg to 50 mg. Trastuzumab may be administered in an amount of 0.5 mg to 10 mg per kg of a body weight. Cetuximab may be administered in an amount of from 100 mg/m² to 500 mg/m² of a surface area of the body.

Vinorelbine may be administered in an amount of 0.5 mg/m² to 50 mg/m² of a surface area of the body. Also, paclitaxel may be administered in an amount of 100 mg/m² to 300 mg/m² of a surface area of the body.

Trastuzumab, sold under the brand name Herceptin™ among others, is a monoclonal antibody used to treat breast cancer. Specifically it is used for breast cancer that is HER2 receptor positive. Trastuzumab is given by slow injection into a vein and injection just under the skin.

Cetuximab is an epidermal growth factor receptor (EGFR) inhibitor used for the treatment of metastatic colorectal cancer, metastatic non-small cell lung cancer and head and neck cancer. Cetuximab is a chimeric (mouse/human) monoclonal antibody given by intravenous infusion that is distributed under the trade name Erbitux™ in the U.S. and Canada by the drug company Bristol-Myers Squibb and outside the U.S. and Canada by the drug company Merck KGaA. In Japan, Merck KGaA, Bristol-Myers Squibb and Eli Lilly have a co-distribution.

Paclitaxel (PTX), sold under the brand name Taxol™ among others, is a chemotherapy medication used to treat a number of types of cancer. This includes ovarian cancer, breast cancer, lung cancer, Kaposi sarcoma, cervical cancer, and pancreatic cancer. It is given by injection into a vein.

In one embodiment, the combination may include poziotinib and a mitotic inhibitor. The mitotic inhibitor may be selected from BT-062, HMN-214, eribulin mesylate, vindesine, EC-1069, EC-1456, EC-531, vintafolide, 2-methoxyestradiol, GTx-230, trastuzumab emtansine (T-DM1), crolibulin, D1302A-maytansinoid conjugates, IMGN-529, lorvotuzumab mertansine, SAR-3419, SAR-566658, IMP-03138, topotecan/vincristine combinations, BPH-8, fosbretabulin tromethamine, estramustine phosphate sodium, vincristine, vinflunine, vinorelbine, RX-21101, cabazitaxel, STA-9584, vinblastine, epothilone A, patupilone, ixabepilone, Epothilone D, paclitaxel, docetaxel, DJ-927, discodermolide, eleutherobin, and pharmaceutically acceptable salts thereof or combinations thereof. An example of the combination may include poziotinib and taxane, vinca alkaloid, or a combination thereof. The vinca alkaloid may be at least one drug selected from the group consisting of vinblastine, vincristine, vindesine and vinorelbine. The taxane may be paclitaxel or docetaxel. An example of the combination may include poziotinib and pacliataxel; or poziotinib and vinorelbine. The neoplasm may be a breast cancer in which Her2 is overexpressed.

In a further embodiment, the combination may include poziotinib and a mitotic inhibitor, such as paclitaxel. In yet another embodiment, the cytotoxic agent may be an EGFR inhibitor, such as a Raf inhibitor, a RAS inhibitor, a MEK inhibitor, a MAPK inhibitor or an ERK inhibitor in combination with poziotinib alone or poziontinib and a second cytotoxic agent.

Poziotinib may be administered in an amount of 0.1 mg to 50 mg. Also, vinorelbine may be administered in an amount of 0.5 mg/m² to 50 mg/m² of a surface area of the body. Also, paclitaxel may be administered in an amount of from 100 mg/m² to 300 mg/m² of a surface area of the body.

Vinorelbine (NVB), sold under the brand name Navelbine™ among others, is a chemotherapy medication used to treat a number of types of cancer. This includes breast cancer and non-small cell lung cancer. It is given by injection into a vein or by mouth. Vinorelbine is in the vinca alkaloid family of medications. It is believed to work by disrupting the normal function of microtubules and thereby stopping cell division.

In one embodiment, the combination may include poziotinib and an mTOR inhibitor, and AKT and/or a P13k system inhibitors. The mTOR inhibitor may be selected from zotarolimus, umirolimus, temsirolimus, sirolimus, sirolimus NanoCrystal™, sirolimus TransDerm™, sirolimus-PNP, everolimus, biolimus A9, ridaforolimus, rapamycin, TCD-10023, DE-109, MS-R001, MS-R002, MS-R003, Perceiva, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, ABTL-0812, and pharmaceutically acceptable salts thereof or combinations thereof. An example of the combination may include poziotinib and rapamycin. The rapamycin may be in the form of an injection. Rapamycin, also known as sirolimus, is a compound produced by the bacterium Streptomyces hygroscopicus.

Poziotinib may be administered in an amount of 0.1 mg to 50 mg. Also, rapamycin may be administered in an amount of 0.5 mg/m² to 10 mg/m² of a surface area of the body.

In one embodiment, the combination may include poziotinib and an antimetabolite. The antimetabolite may be selected from the group consisting of capecitabine, 5-fluorouracil, gemcitabine, pemetrexed, methotrexate, 6-mercaptopurine, cladribine, cytarabine, doxifludine, floxuridine, fludarabine, hydroxycarbamide, decarbazine, hydroxyurea, and asparaginase. An example of the combination may include poziotinib and 5-fluorouracil. The 5-fluorouracil may be in the form of an injection.

Poziotinib may be administered in an amount of 0.1 mg to 50 mg. 5-Fluorouracil may be administered in an amount of 100 mg/m² to 3,000 mg/m² of a surface area of the body.

Fluorouracil (5-FU), sold under the brand name Adrucil™ among others, is a medication used to treat cancer. By injection into a vein it is used for colon cancer, esophageal cancer, stomach cancer, pancreatic cancer, breast cancer, and cervical cancer. As a cream it is used for basal cell carcinoma. Fluorouracil is in the antimetabolite and pyrimidine analog families of medications. How it works is not entirely clear but it is believed to involve blocking the action of thymidylate synthase and thus stopping the production of DNA.

In one embodiment, the combination may include poziotinib and a platinum-based antineoplastic drug. The platinum-based antineoplastic drug may be selected from the group consisting of cisplatin, carboplatin, dicycloplatin, eptaplatin, lobaplatin, miriplatin, nedaplatin, oxaliplatin, picoplatin, and satraplatin. An example of the combination may include poziotinib and cisplatin. The cisplatin may be in the form of an injection.

Poziotinib may be administered in an amount of 0.1 mg to 50 mg. Cisplatin may be administered in an amount of 1 mg/m² to 100 mg/m² of a surface area of the body.

Cisplatin is a chemotherapy medication used to treat a number of cancers. This includes testicular cancer, ovarian cancer, cervical cancer, breast cancer, bladder cancer, head and neck cancer, esophageal cancer, lung cancer, mesothelioma, brain tumors and neuroblastoma. It is used by injection into a vein. Cisplatin is in the platinum-based antineoplastic family of medications. It works in part by binding to DNA and inhibiting DNA replication.

The combination may further include at least one pharmaceutically acceptable carrier. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing the drug combination—poziotinib or its pharmaceutically acceptable solvate or a salt thereof in combination with a cytotoxic agent and/or a molecularly targeted agent, with said cytotoxic agent and/or a molecularly targeted agent encompassing pharmaceutically acceptable salts or solvates thereof—with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmaceutical arts.

Poziotinib in combination with a cytotoxic agent and/or a molecularly targeted agent may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that all the component compounds may be compounded together in a pharmaceutical composition/formulation. Suitably, all the component compounds are administered in separate pharmaceutical compositions.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, suitably, may be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. One or more of flavoring agent, preservative, dispersing agent and coloring agent can also be present.

It should be understood that in addition to the ingredients mentioned above, the formulations may include other agents conventional in the art having regard to the type of formulation in question, for example those suitable for oral administration may include flavoring agents.

In another aspect, therapeutically effective amounts of the combinations of the invention (poziotinib or a pharmaceutically acceptable solvate or a salt thereof in combination with a cytotoxic agent or its pharmaceutically acceptable salt or a solvate thereof and/or a molecularly targeted agent or its pharmaceutically acceptable salt or a solvate thereof) are administered to a human in a regimen simultaneously or consecatively. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attending physician.

Suitably, the present invention relates to treating a neoplasm associated overexpression or amplification of at least one gene of HER1, HER2, and HER4 that is either wild type or mutant. This includes patients who are wild type for one of HER1, HER2, and HER4 and mutant for two of HER1, HER2, and HER4, wild type for two of HER1, HER2, and HER4 and mutant for one of HER1, HER2, and HER4, and mutant for all of HER1, HER2, and HER4.

The term “wild type” as used herein is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand. Neoplasm including cancers that are either wild type or mutant for HER1, HER2, or HER4 or have amplification of HER1, HER2, or HER4 genes or have over expression of HER1, HER2, or HER4 protein are identified by known methods.

For example, wild type or mutant HER1, HER2, and HER4 tumor cells can be identified by DNA amplification and sequencing techniques, DNA and RNA detection techniques, including, but not limited to Northern and Southern blot, respectively, and/or various biochip and array technologies or in-situ hybridization. Wild type and mutant polypeptides can be detected by a variety of techniques including, but not limited to immunodiagnostic techniques such as ELISA, Western blot or immunocytochemistry.

According to another embodiment, provided is a use of the combination drug in preparation of a medicament or medicaments for treating a neoplasm associated with overexpression or amplification of at least one gene of HER1, HER2, and HER4 or a mutant of HER1, HER2, or HER4 in a subject.

According to another embodiment, provided is a method of treating a neoplasm associated with overexpression or amplification of HER1, HER2, or HER4 or a mutant of HER1, HER2, or HER4 in a subject, the method including administrating a combination of active ingredients including therapeutically effective amounts of poziotinib and at least one selected from the group consisting of a cytotoxic agent and a molecularly targeted agent. Descriptions about the combination and the neoplasm associated with overexpression or amplification of HER1, HER2, or HER4 or a mutant of HER1, HER2, or HER4 are the same as defined in the descriptions above.

In a specific embodiment the neoplasm is selected from the group consisting of:

(i) non-small cell lung cancer carrying one or more EGFR mutations selected from L858R substitution, T790M substitution and/or deletion in exon 19;
(ii) estrogen receptor-negative breast cancer with overexpression of HER1 and/or HER2;
(iii) estrogen receptor- and progesterone receptor-double positive breast cancer with HER2 being expressed but without overexpression;
(iv) trastuzumab-resistant breast cancer with overexpression of HER2;
(v) HER1-overexpressing breast cancer triply negative with respect to HER2, progesterone receptor and estrogen receptor;
(vi) esophageal cancer with overexpression of HER2;
(vii) gastric cancer with overexpression of HER2;
(viii) non-small cell lung cancer carrying one or more EGFR mutations comprise one or more point mutations, insertions, and/or deletions of 3-18 nucleotides between amino acids 763-778 or have 2, 3, or 4 EGFR exon 20 mutations at one or more residues selected from the group consisting of A763, A767, S768, V769, D770, N771, P772, and H773 and/or not have an EGFR mutation at residue C797.
(ix) non-small cell lung cancer carrying one or more exon 20 mutations that are selected from the group consisting of A763insFQEA, A767insASV, S768dupSVD, V769insASV, D770insSVD, D770insNPG, H773insNPH, N771del insGY, N771del insFH, and N771dupNPH.

In a further specific embodiment, the combination and the neoplasm to be treated are selected from the group consisting of:

(1) poziotinib and paclitaxel for treating
(a) non-small cell lung cancer carrying EGFR mutation of L858R substitution, T790M substitution and/or deletion in exon 19,
(b) estrogen receptor-negative breast cancer with overexpression of HER2, or
(c) trastuzumab-resistant breast cancer with overexpression of HER2;
   (2) poziotinib and cisplatin for treating
(a) non-small cell lung cancer carrying EGFR mutation of L858R substitution, T790M substitution and/or deletion in exon 19,
(b) estrogen receptor-negative breast cancer with overexpression of HER2, or
(c) trastuzumab-resistant breast cancer with overexpression of HER2;
   (3) poziotinib and 5-fluorouracil for treating
(a) estrogen receptor-negative breast cancer with overexpression of HER2,
(b) trastuzumab-resistant breast cancer with overexpression of HER2; or
(c) esophageal cancer with overexpression of HER2;
   (4) poziotinib and cetuximab for treating non-small cell lung cancer carrying EGFR substitutions of L858R and T790M with overexpression of HER1;
   (5) poziotinib and trastuzumab for treating
(a) trastuzumab-resistant breast cancer with overexpression of HER2, or
(b) gastric cancer with overexpression of HER2; and
   (6) poziotinib and vinorelbine for treating
(a) estrogen receptor-negative breast cancer with overexpression of HER2,
(b) estrogen receptor- and progesterone receptor-double positive and trastuzumab-resistant breast cancer with overexpression of HER2,
(c) estrogen receptor- and progesterone receptor-double positive breast cancer doubly negative with respect to overexpression of HER1 and HER2, or
(d) HER1-overexpressing breast cancer triply negative with respect to HER2, estrogen receptor and progesterone receptor

Also, contemplated herein is a method of treating cancer using a combination of the invention where the constituent drugs of the drug combination are administered in the form of pro-drugs. Pharmaceutically acceptable pro-drugs of the compounds of the invention are readily prepared by those of skill in the art.

When referring to a dosing protocol, the term “day”, “per day” and the like, refer to a time within one calendar day which begins at midnight and ends at the following midnight.

By the term “treating” and derivatives thereof as used herein, is meant therapeutically effective regimens to patients in need thereof. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” as used herein is meant either, simultaneous administration, or any manner of separate sequential administration of therapeutically effective amounts of the constituent drugs—poziotinib in combination with a cytotoxic agent and/or a molecularly targeted agent—or the pharmaceutically acceptable salts or solvates thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

Suitably the combinations of this invention are administered within a “specified period”.

By the term “specified period” and derivatives thereof, as used herein is meant the interval of time between the administration of one of the constituent drug of the inventive combination and another constituent drug. Unless otherwise defined, the specified period can include simultaneous administration. In an embodiment of a two constituent drug combination, when both compounds of the invention are administered once a day the specified period refers to timing of the administration of poziotinib and the other, in the relevant order during a single day. When one or both compounds of the invention are administered more than once a day, the specified period is calculated based on the first administration of each compound on a specific day. All administrations of a compound of the invention that are subsequent to the first during a specific day are not considered when calculating the specific period.

Suitably, if the compounds are administered within a “specified period” and not administered simultaneously, they are both administered within about 24, 12, 11, 10, 9, 8, 7, 6, 5, 4, 3, 2, or 1 hour(s) of each other—in this case, the specified period will be about 24 12, 11, 10, 9, 8, 7, 6, 5, 4, 3, 2, or 1 hour(s). As used herein, for an embodiment of a two constituent drug combination, the administration of poziotinib and the other constituent drug in less than about 45 minutes apart is considered simultaneous administration.

Suitably, when the combination of the invention is administered for a “specified period”, the compounds will be co-administered for a “duration of time”.

By the term “duration of time” and derivatives thereof, as used herein is meant that both compounds of the invention are administered within a “specified period” for an indicated number of consecutive days, optionally followed by a number of consecutive days where only one of the component compounds is administered.

Regarding “specified period” administration, in at least one embodiment, during the course of treatment, both compounds will be administered within a specified period for at least 1, 2, 3, 5, 7, 14, or 30 day(s)—in this case, the duration of time will be at least 1, 2, 3, 5, 7, 14, or 30 day(s). When, during the course of treatment, both compounds are administered within a specified period for over 30 days, the treatment is considered chronic treatment and will continue until an altering event, such as a reassessment in cancer status or a change in the condition of the patient, warrants a modification to the protocol.

Further regarding “specified period” administration, in another embodiment, during the course of treatment with a two constituent drug combination both constituent drugs will be administered within a specified period for at least 1 day, followed by the administration of poziotinib alone for at least 1, 2, 3, 4, 5, 6, or 7 day(s)—in this case, the duration of time will be at least 2, 3, 4, 5, 6, 7, or 8 days; suitably, during the course of treatment, both compounds will be administered within a specified period for at least 2 consecutive days, followed by administration of poziotinib alone for at least 1 day, 2, 3, 4, 5, 6, or 7 consecutive days—in this case, the duration of time will be at least 3, 4, 5, 6, 7, 8, or 9 days; suitably, during the course of treatment, both compounds will be administered within a specified period for at least 3 consecutive days, followed by administration of poziotinib alone for at least 1 day, 2, 3, 4, 5, 6, or 7 consecutive days—in this case, the duration of time will be at least 4, 5, 6, 7, 8, 9, or 10 days; suitably, during the course of treatment, both compounds will be administered within a specified period for at least 4 consecutive days, followed by administration of poziotinib alone for at least 1 day, 2, 3, 4, or 7 consecutive days—in this case, the duration of time will be at least 5, 6, 7, 8 or 11 days; suitably, during the course of treatment, both compounds will be administered within a specified period for at least 5 consecutive days, followed by administration of poziotinib alone for at least 1 day, 2, 3, 4, or 5 consecutive days—in this case, the duration of time will be at least 6, 7, 8, 9, or 10 days. In another embodiment, during the course of treatment, both compounds will be administered within a specified period for from 1 to 3 consecutive days, followed by administration of poziotinib alone for from 3 to 7 consecutive days.

In another embodiment, during the course of treatment, both compounds will be administered within a specified period for from 3 to 6 consecutive days, followed by administration of poziotinib alone for from 1 to 4 consecutive days. Suitably, during the course of treatment, both compounds will be administered within a specified period for 2 consecutive days, followed by administration of poziotinib alone for from 3 to 7 consecutive days. Suitably, during the course of treatment, both compounds will be administered within a specified period for from 1 to 3 days over a 7 day period, and during the other days of the 7 day period poziotinib will be administered alone. In yet another embodiment, during the course of treatment, both compounds will be administered within a specified period for 2 days over a 7 day period, and during the other days of the 7 day period poziotinib will be administered alone.

Further regarding “specified period” administration, during the course of treatment, for an embodiment of a two constituent drug combination both compounds will be administered within a specified period for at least 1 day, followed by the administration of the other constituent drug alone for at least 1, 2, 3, 4, 5, 6, or 7 day(s)—in this case, the duration of time will be at least 2, 3, 4, 5, 6, 7, or 8 days; suitably, during the course of treatment, both compounds will be administered within a specified period for at least 2 consecutive days, followed by administration of the other constituent drug alone for at least 1 day, 2, 3, 4, 5, 6, or 7 consecutive days—in this case, the duration of time will be at least 3, 4, 5, 6, 7, 8, or 9 days; suitably, during the course of treatment, both compounds will be administered within a specified period for at least 3 consecutive days, followed by administration of the other constituent drug alone for at least 1 day, 2, 3, 4, 5, 6, or 7 consecutive days—in this case, the duration of time will be at least 4, 5, 6, 7, 8, 9, or 10 days; suitably, during the course of treatment, both compounds will be administered within a specified period for at least 4 consecutive days, followed by administration of the other constituent drug alone for at least 1 day, 2, 3, 4, or 7 consecutive days—in this case, the duration of time will be at least 5, 6, 7, 8 or 11 days; suitably, during the course of treatment, both compounds will be administered within a specified period for at least 5 consecutive days, followed by administration of the other constituent drug alone for at least 1 day, 2, 3, 4, or 5 consecutive days—in this case, the duration of time will be at least 6, 7, 8, 9, or 10 days. Suitably, during the course of treatment, both compounds will be administered within a specified period for from 1 to 3 consecutive days, followed by administration of the other constituent drug alone for from 3 to 7 consecutive days. Suitably, during the course of treatment, both compounds will be administered within a specified period for from 3 to 6 consecutive days, followed by administration of the other constituent drug alone for from 1 to 4 consecutive days. Suitably, during the course of treatment, both compounds will be administered within a specified period for 2 consecutive days, followed by administration of the other constituent drug alone for from 3 to 7 consecutive days. Suitably, during the course of treatment, both compounds will be administered within a specified period for from 1 to 3 days over a 7 day period, and during the other days of the 7 day period the other constituent drug will be administered alone. Suitably, during the course of treatment, both compounds will be administered within a specified period for 2 days over a 7 day period, and during the other days of the 7 day period the other constituent drug will be administered alone.

Further regarding “specified period” administration, during the course of treatment, for an embodiment of a two constituent drug combination, poziotinib and the other constituent drug will be administered within a specified period for from 1 to 3 days over a 7 day period, and during the other days of the 7 day period poziotinib will be administered alone. Suitably, this 7 day protocol is repeated for 2 cycles or for 14 days; suitably for 4 cycles or 28 days; suitably for continuous administration.

Suitably, during the course of treatment, for an embodiment of a two constituent drug combination, poziotinib and the other constituent drug will be administered within a specified period for from 1 to 3 days over a 7 day period, and during the other days of the 7 day period the other constituent drug will be administered alone. Suitably, this 7 day protocol is repeated for 2 cycles or for 14 days; suitably for 4 cycles or 28 days; suitably for continuous administration.

Suitably, during the course of treatment, for an embodiment of a two constituent drug combination, poziotinib and the other constituent drug will be administered within a specified period for from 1 to 5 days over a 14 day period, and during the other days of the 14 day period poziotinib will be administered alone. Suitably, this 14 day protocol is repeated for 2 cycles or for 28 days; suitably for continuous administration.

Suitably, during the course of treatment, for an embodiment of a two constituent drug combination, poziotinib and the other constituent drug will be administered within a specified period for from 1 to 5 days over a 14 day period, and during the other days of the 14 day period the other constituent drug will be administered alone. Suitably, this 14 day protocol is repeated for 2 cycles or for 28 days; suitably for continuous administration.

Suitably, if the compounds are not administered during a “specified period”, they are administered sequentially. By the term “sequential administration” as used herein is meant for example for an embodiment of a two constituent drug combination, that one of poziotinib and the other constituent drug is administered for 1 or more consecutive days and the other of poziotinib and the other constituent drug is subsequently administered for 1 or more consecutive days. Unless otherwise defined, the “sequential administration” and in all dosing protocols described herein in the case of a two constituent drug combination, do not have to commence with the start of treatment and terminate with the end of treatment, it is only required that the administration of one of poziotinib and the other constituent drug followed by the administration of the other, or the indicated dosing protocol, occur at some point during the course of treatment. Also, contemplated herein is a drug holiday utilized between the sequential administration of one of poziotinib and the other constituent drug and the other. As used herein, a drug holiday is a period of days after the sequential administration of one of poziotinib and the other constituent drug and before the administration of the other where neither poziotinib nor the other constituent drug is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

Regarding Sequential Administration:

Suitably, for example for an embodiment of a two constituent drug combination, one of poziotinib and the other constituent drug is administered for from 1 to 30 consecutive days, followed by an optional drug holiday, followed by administration of the other for from 1 to 30 consecutive days.

Suitably, for example for an embodiment of a two constituent drug combination, the other constituent drug will be administered first in the sequence, followed by an optional drug holiday, followed by administration of poziotinib.

Suitably, poziotinib will be administered first in the sequence, followed by an optional drug holiday, followed by administration of the other constituent drug.

It is understood that a “specified period” administration and a “sequential” administration can be followed by one or more cycles of repeat dosing or can be followed by an alternate dosing protocol, and a drug holiday may precede the repeat dosing or alternate dosing protocol.

Suitably, the amount of poziotinib administered as part of the combination according to the present invention will be an amount selected from about 0.1 mg to about 50 mg; suitably, the amount will be selected from about 0.5 mg to about 50 mg; suitably, the amount will be selected from about 1 mg to about 50 mg; suitably, the amount will be selected from about 5 mg to about 50 mg; suitably, the amount will be selected from about 1 mg to about 30 mg; suitably, the amount will be selected from about 5 mg to about 20 mg; suitably, the amount will be selected from about 1 mg to about 10 mg; suitably, the amount will be selected from about 0.1 mg to about 5 mg; suitably, the amount will be 1 mg, suitably, the amount will be 5 mg, suitably, the amount will be 10 mg, suitably, the amount will be 20 mg, suitably, the amount will be 30 mg; suitably, the amount will be 50 mg. Accordingly, the amount of poziotinib administered as part of the combination according to the present invention will be an amount selected from about 0.1 mg to about 50 mg. For example, the amount of poziotinib administered as part of the combination according to the present invention is suitably selected from 1 mg, 5 mg, 10 mg, 20 mg, 30 mg and 50 mg. Suitably, the selected amount of poziotinib is administered from 1 to 4 times a day, in one or more tablets. Suitably, the selected amount of poziotinib is administered twice a day, in one or more tablets. Suitably, the selected amount of poziotinib is administered once a day, in one or more tablets. Suitably, the administration of poziotinib will begin as a loading dose. Suitably, the loading dose will be an amount from 2 to 100 times the maintenance dose; suitably from 2 to 10 times; suitably from 2 to 5 times; suitably 2 times; suitably 3 times; suitably 4 times; suitably 5 times. Suitably, the loading does will be administered from 1 to 7 days; suitably from 1 to 5 days; suitably from 1 to 3 days; suitably for 1 day; suitably for 2 days; suitably for 3 days, followed by a maintenance dosing protocol.

Suitably, the amount of the second constituent drug that is not poziotinib administered as part of the combination according to the present invention will be an amount selected from about 0.1 mg to about 3,500 mg/m², from about 0.5 mg to about 3,500 mg/m²; suitably, the amount will be selected from about 1.0 mg to about 3,500 mg/m²; suitably, the amount will be selected from about 10.0 mg to about 3,500 mg/m²; suitably, the amount will be selected from about 50.0 mg to about 3,500 mg/m²; suitably, the amount will be selected from about 100.0 mg to about 3,500 mg/m²; suitably, the amount will be selected from about 100.0 mg to about 3,000 mg/m²; suitably, the amount will be selected from about 0.1 mg to about 20 mg/m²; suitably, the amount will be selected from about 0.5 mg to about 10 mg/m²; suitably, the amount will be selected from about 0.5 mg to about 10 mg/kg; suitably, the amount will be selected from about 0.5 mg to about 50 mg/m²; suitably, the amount will be selected from about 50 mg to about 1,000 mg/m²; suitably, the amount will be selected from about 100 mg to about 500 mg/m²; suitably, the amount will be selected from about 100 mg to about 300 mg/m²; Suitably, the selected amount of the second constituent drug that is not poziotinib is administered from 1 to 4 times a day. Suitably, the selected amount of the second constituent drug is administered 1 to 4 times a day.

As used herein, all amounts specified for poziotinib and the other constituent drug(s) are indicated as the administered amount of free or unsalted and unsolvated compound per dose.

Accordingly, the invention further provides pharmaceutical compositions, which include poziotinib and the other constituent drug(s), and one or more pharmaceutically acceptable carriers.

According to another embodiment, provided is a kit for treating a neoplasm associated with overexpression or amplification of HER1, HER2, or HER4 or a mutant of HER1, HER2, or HER4 in a subject, the kit including a first part and a second part, wherein the first part includes poziotinib, and the second part includes at least one active ingredient selected from the group consisting of a cytotoxic agent and a molecularly targeted agent. The kit can also further comprise a package insert comprising instructions for treating a neoplasm associated with overexpression or amplification of HER1, HER2, or HER4 or a mutant thereof in a subject.

Descriptions of poziotinib, the cytotoxic agent, and the molecularly targeted agent and expressions “HER1, HER2, or HER4 or a mutant of HER1, HER2, or HER4” and “overexpression” are the same as defined in the descriptions above.

By the term “combination kit” as used herein is meant the pharmaceutical composition or compositions that are used to administer the drugs. For example, poziotinib, or a pharmaceutically acceptable solvate and/or salt thereof, is associated with at least one chosen from cytotoxic agents and molecularly targeted agents, including the pharmaceutically acceptable salts or solvates of the cytotoxic agents and molecularly targeted agents as well. When the compounds can be administered simultaneously, at least two of the compounds can be in a single pharmaceutical composition, such as a tablet. When the compounds are not administered simultaneously, the combination kit will contain poziotinib and the other constituent drug(s) or pharmaceutically acceptable salts or solvates thereof, in separate pharmaceutical compositions. The combination kit can comprise poziotinib and the other constituent drug(s) or pharmaceutically acceptable salts or solvates thereof in separate pharmaceutical compositions in a single package or in separate pharmaceutical compositions in separate packages.

In the combination kit, the components may be provided in a form which is suitable for sequential, separate and/or simultaneous administration.

The “combination kit” can also be provided with instruction, such as dosage and administration instructions. Such dosage and administration instructions can be of the kind that is provided to a doctor, for example by a drug product label, or they can be of the kind that is provided by a doctor, such as instructions to a patient.

According to another embodiment, a method for treating a neoplasm associated with overexpression or amplification of HER1, HER2, or HER4 or a mutant of HER1, HER2, or HER4 in a subject in need thereof is described, wherein prior to the administration of the suitable combination therapies described herein, the genotypic and/or phenotypic status of the subject's EGFR such as HER1, HER2, HER4 are determined. In yet another embodiment, such status of subject's HER1, HER2 and HER4 maybe determined by suitable immunohistochemistry or in-situ rehybridization methodologies. In yet another embodiment, the present invention also provides for a method of treating a human patient at risk of developing a condition associated with overexpression or amplification of HER1, HER2, or HER4 or a mutant of HER1, HER2, or HER4 by first determining the predisposition of the such human patient individual to HER1, HER2 or HER4 mutation and then administering therapeutically effective combinations described herein.

One or more embodiments will now be described in more detail with reference to the following examples. However, these examples are not intended to limit the scope of the one or more embodiments.

### Example 1: Cell Growth Inhibition Assay and Combination Data Analysis

The effect of the inventive compositions on the cancer cell growth inhibition was conducted in EGFR-overexpressed or -mutated cancer cell lines.

1. Cell Lines and Cell Growth Conditions and the IC50 Determination of Individual Compounds and Combinations Thereof

The information of cancer cells and cell growth conditions are shown in Table 1, below.

TABLE 1

| | Cell | Gene | | | Cell Culture |
|---|---|---|---|---|---|
| Cancer | Line | status | Distributor | Cat. # | Condition |
| NSCLC | HCC827 | Her1 | ATCC | CRL2868 | RPMI1640, |
| | | exon19del | | | 10% FBS |
| | H1975 | Her1 | ATCC | CRL5908 | RPMI1640, |
| | | T790M/ | | | 10% FBS |
| | | L858R | | | |
| | Calu-3 | | | | |
| Breast | BT-474 | HER2+, | ATCC | HT-20 | HybriCare |
| | | ER−, PR+, | | | Medium, |
| | | mPIK3CA | | | 10% FBS |
| | MDA-MB-361 | HER2+, | ATCC | HTB-27 | L-15, 20% |
| | | ER+, PR+, | | | FBS |
| | | mPIK3CA | | | |
| | MCF7 | ER/PR+, | ATCC | HTB-22 | RPMI1640, |
| | | mPIK3CA | | | 10% FBS |
| | SK-BR-3 | HER2+ | ATCC | HTB-30 | RPMI1640, |
| | | | | | 10% FBS |
| | MDA-MB-468 | HER1+, | ATCC | HTB-132 | L-15, 16% |
| | | Her2/ER/ | | | FBS |
| | | PR−, | | | |
| | | mPTEN | | | |
| | MDA-MB-453 | HER2+, | ATCC | HTB-131 | L-15, 10% |
| | | mPIK3CA/ | | | FBS |
| | | PTEN | | | |
| Esopha- | TE-4 | HER2+ | ATCC | HB207 | RPMI1640, |
| geal | | | | | 10% FBS |
| Gastric | N87 | HER2+ | ATCC | CRL5822 | RPMI1640, |
| | | | | | 10% FBS |

### Abbreviations

ER: estrogen receptor; PR: progesterone receptor; HER2: human epidermal growth factor receptor 2; HER1: human epidermal growth factor receptor 1; PIK3CA: phosphoinositide-3-kinase, catalytic, alpha polypeptide; PTEN: Phosphatase and tensin homolog; +: positive (overexpression); −: negative; m: mutant; ATCC: American Type Culture Collection; FBS: fetal bovine serum.

All cells except for H1975 were cultured for 72 hours prior to cell plating. Cells were seeded in a 96-well plate at 4 to 15×10³ cells/well. Approximately 24 hours after plating, cells were exposed to ten, two-fold or three fold serial dilutions of test individual compounds or the combination of the two test compounds at a constant molar to molar ratio or non-constant ratio of two agents. Cells were incubated in the presence of compounds for 3 days except for H1975, which was for 2 days at 3×10³ cells/well.

2. Measurement of Cell Growth Inhibition (CI)

Cell growth inhibition based on measurement of cellular protein content is determined by sulforhodamine B colorimetric assay according to the “Nat Protoc. 2006; 1(3), 1112-6”. The absorbance was read on a SpectraMax™ plate reader at 540 nm. For H1975 cells, cell growth (%) is determined by adding CellTiter™ 96 Aqueous One Solution Cell Proliferation Assay reagent (Promega) according to the manufacturer's protocol.

Inhibition of cell growth was estimated after treatment with test agent or combination of test agents for incubating time (2-3 days) and comparing the signal to cells treated with vehicle (DMSO). Cell growth (%) was calculated by following equations.

Percentage growth inhibition was calculated using:

a. [(Ti−Tz)/(C−Tz)]×100, for concentrations for which Ti=Tz

b. [(Ti−Tz)/Tz]×100, for concentrations for which Ti<Tz

Each T value means an absorbance. Tz: Time zero (cell density group before the treatment of test group), C:100% control growth with test agent, Ti: Test growth in the presence of drug at the concentration levels. GI₅₀, the concentration at which 50% inhibition occurs, was calculated from [(Ti−Tz)/(C−Tz)]×100=50.

Combination effects on potency were evaluated by combination Index using Calcusyn™ Software. The range of Combination Index (CI) for two compounds is described in Table 2.

TABLE 2

| Range of CI | |
|---|---|
| (Combination Index) | Description |
| <0.1 | Very strong |
| | synergism |
| 0.1-0.3 | Strong synergism |
| 0.3-0.7 | Synergism |
| 0.7-0.85 | Moderate synergism |
| 1.1-1.2 | Slight synergism |
| 0.9-1.1 | Nearly additive |
| 1.1-1.2 | Slight antagonism |
| 1.2-1.45 | Moderate |
| | Antagonism |
| 1.45-3.3 | Antagonism |
| 3.3-10 | Strong antagonism |
| >10 | Very strong |
| | antagonism |

3. Results

The growth inhibition effect of the each single agent and the combination effect (CI) for in vitro cell growth are summarized in Table 3a-3f.

TABLE 3a

| Cell lines | Single or combination effect | | | |
|---|---|---|---|---|
| HCC827 | Drugs | Poziotinib | Paclitaxel | CI |
| | Single agent, GI₅₀ (nM) | 0.4 | 3.4 | — |
| | Combination, | 0.05 | 0.5 | 0.5 |
| | Drug concentration | 0.1 | 1 | 0.6 |
| | (nM) | 0.2 | 2.5 | 0.85 |
| | Drugs | Poziotinib | Cisplatin | CI |
| | Single agent, GI₅₀ (nM) | 0.4 | >20,000 | — |
| | Combination, | 0.05 | 100 | 0.22 |
| | Drug concentration | 0.1 | 200 | 0.4 |
| | (nM) | 0.2 | 400 | 0.4 |
| | | 0.5 | 1,000 | 0.3 |
| | Drugs | Poziotinib | 5-FU | CI |
| | Single agent, GI₅₀ (nM) | 0.4 | 980 | — |
| | Combination, | 0.1 | 200 | >1.2 |
| | Drug concentration | 0.2 | 400 | 0.9 |
| | (nM) | 0.5 | 1,000 | >1.2 |
| | | 1 | 2,000 | >1.2 |

TABLE 3b

| Cell lines | Single or combination effect | | | |
|---|---|---|---|---|
| H1975 | Drugs | Poziotinib | Pactitaxel | CI |
| | Single agent, GI₅₀ | 1.3 | 21.6 | — |
| | (nM) | | | |
| | Combination, | 0.08 | 0.78 | >1.2 |
| | Drug concentration | 0.16 | 1.56 | 0.7 |
| | (nM) | 0.31 | 3.13 | 0.9 |
| | | 0.63 | 6.25 | >1.2 |
| | Drugs | Poziotinib | Cisplatin | CI |
| | Single agent, GI₅₀ | 1.3 | >10,000 | — |
| | (nM) | | | |
| | Combination, | 0.16-5 | 10,000 | >1.2 |
| | Drug concentration | | | |
| | (nM) | | | |
| | Drugs | Poziotinib | 5-FU | CI |
| | Single agent, GI₅₀ | 2.6 | >10,000 | — |
| | (nM) | | | |
| | Combination, | 0.3125 | 10,000 | >1.2 |
| | Drug concentration | 0.625 | | 0.5 |
| | (nM) | 1.25 | | 0.9 |
| | | 2.5 | | >1.2 |
| | Drugs | Poziotinib | Cetuximab | CI |
| | Single agent, GI₅₀ | 3 | >10,000 | — |
| | (nM) | | | |
| | Combination, | 0.391 | 10,000 | 0.749 |
| | Drug concentration | 0.781 | | 0.28 |
| | (nM) | 1.563 | | 0.314 |
| | | 3.125 | | 0.406 |
| | | 6.25 | | 0.651 |
| | | 12.5 | | 1.074 |
| | | 25 | | 2.139 |
| | | 50 | | 4.402 |
| | | 0.391 | 78.12 | 0.036 |
| | | 0.781 | 156.25 | 0.074 |
| | | 1.563 | 312.5 | 0.159 |
| | | 3.125 | 625 | 0.429 |
| | | 6.25 | 1,250 | 1.08 |
| | | 12.5 | 2,500 | 2.743 |
| | | 25 | 5,000 | 8.05 |

TABLE 3c

| Cell lines | Single or combination effect | | | |
|---|---|---|---|---|
| BT-474 | Drugs | Poziotinib | Paclitaxel | CI |
| | Single agent, GI₅₀ | 0.5 | 1 | — |
| | Combination, | 0.2 | 0.2 | 0.7 |
| | Drug concentration | 0.5 | 0.5 | <0.1 |
| | (nM) | 1 | 1 | <0.1 |
| | | 2 | 2 | 0.4 |
| | Drugs | Poziotinib | Cisplatin | CI |
| | Single agent, GI₅₀ | 0.5 | >20,000 | — |
| | (nM) | | | |
| | Combination, | 0.2 | 10,000 | <0.1 |
| | Drug concentration | 6.5 | | <0.1 |
| | (nM) | 1 | | 0.2 |
| | | 2 | | <0.1 |
| | | 0.2 | 1,000 | 0.7 |
| | | 0.5 | 2,500 | 0.5 |
| | | 1 | 5,000 | 0. |
| | | 2 | 10,000 | 0.4 |
| | Drugs | Poziotinib | 5-FU | CI |
| | Single agent, GI₅₀ | 0.5 | 9,000 | — |
| | (nM) | | | |
| | Combination, | 0.2 | 10,000 | <0.1 |
| | Drug concentration | 0.5 | | <0.1 |
| | (nM) | 1 | | <0.1 |
| | | 2 | | <0.1 |
| | Drugs | Poziotinib | Trastuzumab | CI |
| | Single agent, GI₅₀ | 0.42 | 82 | — |
| | (nM) | | | |
| | Combination, | 0.02 | 2 | 0.94 |
| | Drug concentration | 0.05 | 5 | 0.85 |
| | (nM) | 0.1 | 10 | 1.2 |
| | | 0.2 | 20 | 1.3 |
| | | 0.5 | 50 | 0.46 |
| | Drugs | Poziotinib | Vinorelbine | CI |
| | Single agent, GI₅₀ | 0.8 | 2.3 | — |
| | (nM) | | | |
| | Combination, | 0.1 | 0.2 | >10 |
| | Drug concentration | 0.5 | 1 | 2.6 |
| | (nM) | 1 | 2 | 1.5 |
| | | 1.5 | 3 | 0.1 |

TABLE 3d

| Cell lines | Single or combination effect | | | |
|---|---|---|---|---|
| MDA-MB-453 | Drugs | Poziatinib | Paclitaxel | CI |
| | Single agent, GI₅₀ | 2.5 | 3 | — |
| | (nM) | | | |
| | Combination, | 0.6 | 0.6 | 0.5 |
| | Drug concentration | 1.25 | 1.25 | 0.9 |
| | (nM) | | | |
| | Drugs | Poziatinib | Cisplatin | CI |
| | Single agent, GI₅₀ | 2.5 | >10,000 | — |
| | (nM) | | | |
| | Combination, | 0.31 | 10,000 | 0.4 |
| | Drug concentration | 0.6 | | 0.7 |
| | (nM) | | | |
| | Drugs | Poziotinib | 5-FU | CI |
| | Single agent, GI₅₀ | 2.5 | 385 | — |
| | (nM) | | | |
| | Combination, | 0.33 | 62.5 | 0.7 |
| | Drug concentration | 0.6 | 125 | 0.4 |
| | (nM) | 1.25 | 250 | 0.7 |
| | | 2.5 | 500 | 0.7 |
| | | 0.625 | 300 | 0.7 |
| | | 1.25 | | 0.9 |
| | Drugs | Poziotinib | Trastuzumab | CI |
| | Single Agent, GI₅₀ | 1.5 | >100 | — |
| | (nM) | | | |
| | Combination, | 0.05 | 0.5 | >1.1 |
| | Drug concentration | 0.1 | 1 | 0.5 |
| | (nM) | 0.5 | 5 | 0.69 |
| | | 1 | 10 | 1.5 |

TABLE 3e

| Cell lines | Sngle or connbination effect | | | |
|---|---|---|---|---|
| SK-BR3 | Drugs | Poziotinib | Vinorelbine | CI |
| | Single agent, GI₅₀ | 0.9 | 1.7 | — |
| | (nM) | | | |
| | Combination, | 0.1 | 0.2 | 3.4 |
| | Drug concentration | 0.5 | 1 | 2.0 |
| | (nM) | 1 | 2 | 1.1 |
| | | 1.5 | 3 | 0.6 |
| MCF-7 | Drugs | Poziotinib | Vinorelbine | CI |
| | Single agent, GI₅₀ | 2,738 | 3.6 | — |
| | (nM) | | | |
| | Combination, | 100 | 2 | 0.9 |
| | Drug concentration | 500 | | 0.8 |
| | (nM) | 1,000 | | 0.7 |
| | | 2,500 | | 0.4 |
| | | 5,000 | | 0.1 |
| | | 10000 | | <0.1 |
| MDA-MB-361 | Drugs | Poziotinib | Vinorelbine | CI |
| | Single agent, GI₅₀ | 21 | 1.1 | — |
| | (nM) | | | |
| | Combination, | 1 | 1 | 2.1 |
| | Drug concentration | 10 | | 1.4 |
| | (nM) | 100 | | 0.8 |
| | | 100 | | 0.8 |
| | | 1000 | | 0.1 |
| MDA-MB-468 | Drugs | Poziotinib | Vinorelbine | CI |
| | Single agent, GI₅₀ | 640 | 0.3 | — |
| | nM | | | |
| | Combination, | 50 | 0.1 | 1.1 |
| | Drug concentration | 100 | | 0.3 |
| | (nM) | 500 | | 0.9 |
| | | 1000 | | 0.6 |

TABLE 3f

| Cell lines | Single or combination effect | | | |
|---|---|---|---|---|
| N87 | Drugs | Poziotinib | Trastuzumab | CI |
| | Single agent, GI₅₀ (nM) | 0.1 | >10,000 | — |
| | Combination | 0.01 | 0.1 | 0.36 |
| | Drug concentration | 0.05 | 0.5 | 1.0 |
| | (nM) | 0.1 | 1 | 1.2 |
| TE-4 | Drugs | Pozotinb | 5-FU | CI |
| | Single agent, GI₅₀ (nM) | 0.8 | 7,904 | — |
| | Combination, | 0.5 | 25,000 | 0.86 |
| | Drug concentration | | 10,000 | 0.55 |
| | (nM) | | 5,000 | 0.50 |
| | | | 1,000 | 0.65 |
| | | | 500 | 0.93 |
| | | | 100 | 0.86 |
| | | | 10 | 0.86 |
| | | 1 | 25,000 | 0.49 |
| | | | 10,000 | 0.64 |
| | | | 5,000 | 0.64 |
| | | | 1,000 | 0.34 |
| | | | 500 | 0.30 |
| | | | 100 | 0.34 |
| | | | 10 | 0.37 |

Fifty percent growth inhibition values (GI₅₀) for combinations of poziotinib with partner drugs were calculated and the single dosing growth inhibition (%) at the dose of combo GI₅₀ were also calculated by using GraphPad Prism™ v6. The combination Index (CI) at the dose of combination GI₅₀ was evaluated by using CalcuSyn™ (Biosoft). The combination effect in vitro cell growth is summarized in Table 4.

TABLE 4
Summary

| | Combination, GI₅₀ | | | |
|---|---|---|---|---|
| | Poziotinib | Combination Drug | | |
| Cell lines | (nM) | (nM) | | CI |
| HCC827 | 0.29 | Paclitaxel | 2.9 | 1.16 |
| | 0.15 | Cisplatn | 300 | 0.09 |
| | 0.21 | 5-FU | 420 | 0.99 |
| H1975 | 0.8 | Paclitaxel | 8 | 2.31 |
| | 1.6 | Cisplatin | 10,000 | 2.32 |
| | 0.5 | 5-FU | 10,000 | 0.40 |
| BT-474 | 1.0 | Paclitaxel | 1.0 | 0.2 |
| | 0.5 | Cisplatin | 2,500 | 0.3 |
| | 1.1 | 5-FU | 5,500 | 0.3 |
| | 0.34 | Trastuzumab | 34 | 0.63 |
| | 0.79 | Vinorelbine | 1.58 | 1.7 |
| MDA-MB-453 | <0.31 | Paclitaxel | <0.31 | N/A |
| | <0.31 | Cisplatin | 10,000 | N/A |
| | 0.24 | Trastuzumab | 2.4 | 0.48 |
| SK-BR-3 | 0.6 | Vinorelbine | 1.2 | 1.58 |
| MCF-F | 224 | Vinorelbine | 2 | 1.0 |
| MDA-MB-361 | 1.2 | Vinorelbine | 1 | 1.9 |
| MDA-MB-468 | 810 | Vinorelbine | 0.1 | 1.0 |
| N87 | 0.035 | Trastuzumab | 0.35 | 0.47 |
| TE-4 | 0.5 | 5-FU | 1100 | 0.87 |

### Example 2: Synergy Effect Study on Cell Proliferation of Selected Cell Lines

In this example, effects of direct cell killing of selected cancer cell line Calu-3, BT-474 and SK-BR-3 treated with Poziotinib alone or in combination with other agents were assessed.

1. Cell Lines and Cell Growth Conditions and the IC50 Determination of Individual Compounds and Combinations Thereof.

All cells were cultured in the media listed in the temperature of 37° C., 5% CO2 and 95% humidity. Cells were seeded in a 96-well plate at 4 to 15×10³ cells/well. Approximately 24 hours after plating, cells were exposed to ten, two-fold or three fold serial dilutions of test individual compounds or the combination of the two test compounds at a constant molar to molar ratio or non-constant ratio of two agents. Cells were incubated in the presence of compounds for 3 days.

The growth inhibition effect of the each individual and combination regimen is provided below herein

TABLE 6a

| Compound | | Calu-3 | BT-474 |
|---|---|---|---|
| Pozotinb | IC50 (μM) | 0.002 | <0.0015 |
| | Max inh. (%) | 89.22% | 68.16% |
| Letrozole | IC50 (μM) | — | >10 |
| | Max inh. (%) | — | −15.80% |
| Palbociclib | IC50 (μM) | — | >5 |
| | Max inh. (%) | — | 12.13% |
| Sorafenib | 1050 (μM) | 4.475 | — |
| | Max inh. (%) | 64.29% | — |
| Temsirolimus | IC50 (μM) | 0.167 | — |
| | Max inh. (%) | 58.19% | — |
| Cisplatin | 1050 (μM) | 4.968 | >100 |
| | Max inh. (%) | 59.75% | 41.78% |

TABLE 6b

| Compound | | Calu-3 | BT-474 |
|---|---|---|---|
| Poziotinib | IC50 (μM) | 0.009 | <0.0015 |
| | Max inh. (%) | 94.51% | 71.74% |
| Trametinib | IC50 (μM) | 0.042 | >10 |
| | Max inh. (%) | 89.69% | 15.91% |
| Pemetrexed | IC50 (μM) | >10 | >10 |
| | Max inh. (%) | 49.78% | 1.78% |
| Cisplatin | IC50 (μM) | 1.749 | >100 |
| | Max inh. (%) | 69.39% | 37.43% |

TABLE 6c

| compound | | Calu-3 | SK-BR-3 |
|---|---|---|---|
| Poziotinib | IC50 (μM) | 0.001 | 0.001 |
| | Max inh. (%) | 81.67% | 57.81% |
| T-DM1 | IC50 (μg/ml) | 90.449 | <0.030 |
| | Max inh. (%) | 55.24% | 85.37% |
| Cisplatin | IC50 (μM) | 7.852 | 0.549 |
| | Max inh. (%) | 68.77% | 98.11% |

The cell inhibitory activity of individual tested compounds representing a variety of mechanisms of action was examined on Calu-3, BT-474 and SK-BR-3 cell lines. Poziotinib exhibited greater potency on cell proliferation inhibition in the three cell lines, with IC50 of 0.001 μM to 0.0090 for Calu-3, less than 0.0015 μM and 0.001 μM for BT-474, and 0.001 μM for SK-BR-3 separately. For Calu-3 cell line, the IC50 of Sorafenib, Temsirolimus, Trametinib were 4.475, 0.167 and 0.042 μM respectively, while BT-474 displayed weak or no response upon treatment of Letrozole, Palbociclib and Trametinib. Pemetrexed was not effective on these two cells, rather T-DM1 was most responsive and the 1050 was 90.449 μg/ml for Calu-3 and less than 0.030 μg/ml for SK-BR-3.

2. Measurement of Cell Growth Inhibition (CI)

Inhibition of cell growth was estimated after treatment with test agent or combination of test agents for incubating time (2-3 days) and comparing the signal to cells treated with vehicle (DMSO). Cell growth (%) was calculated by following equations.

Percentage growth inhibition was calculated using both Bliss independence model and Lowewe additivity model. A score higher than 5 indicates synergy and a score less that −5 indicates antagonism.

TABLE 5

| Range of CI | |
|---|---|
| (Combination Index) | Description |
| <0.1 | Very strong |
| | synergism |
| 0.1-0.3 | Strong synergism |
| 0.3-0.7 | Synergism |
| 0.7-0.85 | Moderate synergism |
| 0.85-0.9 | Slight synergism |
| 0.9-1.1 | Nearly additive |
| 1.1-1.2 | Slight antagonism |
| 1.2-1.45 | Moderate |
| | Antagonism |
| 1.45-3.3 | Antagonism |
| 3.3-10 | Strong antagonism |
| >10 | Very strong |
| | antagonism |

3. Results

Calu-3, BT-474 or SK-BR-3 cell lines were tested with Poziotinib combined with a second compound respectively in a 6×6 combo matrix for 3 days at 37° C., 5% CO2 and 95% humidity as shown in combinations shown in the tables herein below:

TABLE 6d

| | | Compound 2 | | |
|---|---|---|---|---|
| Assay | Compound 1 | For Calu-3 | For BT-474 | For SK-BR-3 |
| 1 | Poziotinib, | Sorafenib, | Letrozole, | — |
| | | Temsirolimus | Palbociclib | |
| 2 | Poziotinib, | Trametinib, | Trametinib, | — |
| | | Pemetrexed | Pemetrexed | |
| 3 | Poziotinib, | T-DM1 | — | T-DM1 |

TABLE 6e

| | | | | Cell |
|---|---|---|---|---|
| | | Tissue | | density |
| No. | Cell line | Origin | Medium | (/Well) |
| E | Calu-3 | Lung | MEM + 10% FBS + NEAA | 6000 |
| | | NSCLC | | |
| 2 | BT474 | breast | DMEM + 10% FBS + 0.01 mg/ml | 4000 |
| 3 | SK-BR-3 | breast | McCoy's 5a + 10% FBS | 5000 |

The following tables display the CI values (or cell survival rate graph) of two-compound combinations on selected cancer cell lines.

TABLE 6 (f)
Calu-3: Poziotinib + Sorafenib

| | Sorafenib (μM) | | | | | |
|---|---|---|---|---|---|---|
| CI value | | 0.63 | 11.25 | 2.50 | 5.00 | 10 |
| Poziotinib | 0.00001 | >10 | >10 | 0.593 | 0.862 | 1.269 |
| (μM) | 0.0001 | 0.345 | 0.366 | 0.56 | 0.831 | 1.268 |
| | 0.001 | 0.155 | 0.253 | 0.46 | 0.778 | 1.245 |
| | 0.1 | 0.482 | 0.535 | 0.72 | 1.013 | 1.444 |
| | 10 | >10 | >10 | >10 | >10 | >10 |

TABLE 6 (g)
Calu-3: Poziotinib + Temsirolimus

| | | Temsirolimus (μM) | | | | |
|---|---|---|---|---|---|---|
| CI value | | 0.001 | 0.01 | 0.10 | 1.00 | 10 |
| Poziotinib | 0.00001 | >10 | 0.556 | 1.07 | 0.601 | 2.829 |
| (μM) | 0.0001 | 4.7 | 0.501 | 0.17 | 0.854 | 8.667 |
| | 0.001 | 0.02 | 0.009 | 0.007 | 0.007 | 0.049 |
| | 0.1 | 0.246 | 0.208 | 0.215 | 0.196 | 0.2 |
| | 10 | >10 | >10 | >10 | >10 | >10 |

TABLE 6 (h)
Calu-3: Poziotinib + Trametinib

| | Trametinib (μM) | | | | | |
|---|---|---|---|---|---|---|
| CI value | | 0.001 | 0.01 | 0.10 | 1.00 | 10 |
| Poziotinib | 0.00001 | 5.896 | 1.457 | 0.362 | 0.815 | 5.183 |
| (μM) | 0.0001 | 0.829 | 0.637 | 0.295 | 0.527 | 3.918 |
| | 0.001 | 0.064 | 0.104 | 0.052 | 0.101 | 1.306 |
| | 0.1 | 0.208 | 0.117 | 0.055 | 0.054 | 0.19 |
| | 10 | 6.552 | 6.783 | 5.708 | 5.669 | 8.098 |

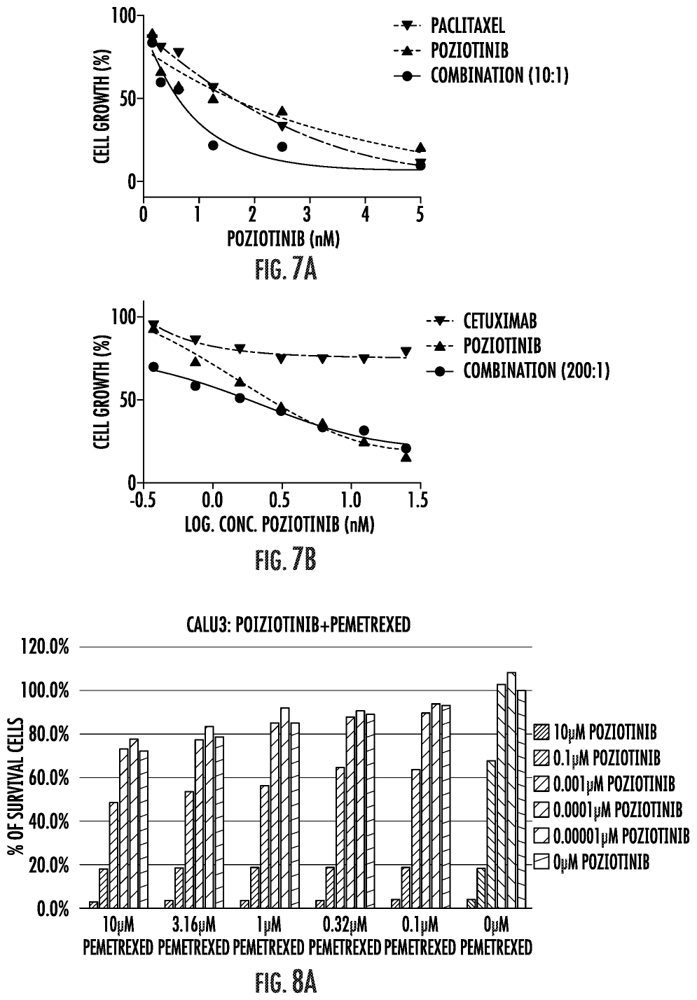

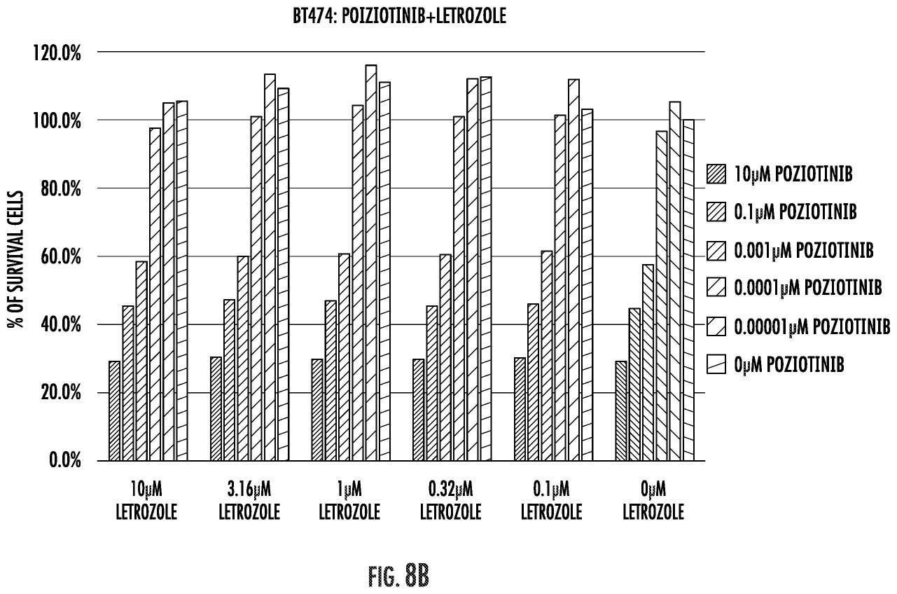

FIGS. 8 (A) and (B) display the effect of Poziotnib and Pemetrexed or Letrozole combination on Calu3 cell lines.

TABLE 6 (i)
Calu-3: Poziotinib + T-DM1

| | T-DM1(μg/ml) | | | | | |
|---|---|---|---|---|---|---|
| CI value | | 0.020 | 0.20 | 2.00 | 20.00 | 200 |
| Poziotinib | 0.0001235 | 1.081 | 0.263 | 0.343 | 0.848 | 1.973 |
| (μM) | 0.0003704 | 1.458 | 0.686 | 0.396 | 0.487 | 1.051 |
| | 0.0011111 | 1.072 | 0.628 | 0.419 | 0.405 | 0.384 |
| | 0.0033333 | 0.596 | 0.484 | 0.434 | 0.309 | 0.283 |
| | 0.01 | 0.874 | 0.73 | 0.656 | 0.583 | 0.432 |

TABLE 6 (j)
BT474: Poziotinib + Palbociclib

| | Palbociclib (μM) | | | | | |
|---|---|---|---|---|---|---|
| CI value | | 0.10 | 0.32 | 1.00 | 3.16 | 10 |
| Poziotinib | 0.00001 | >10 | >10 | >10 | >10 | 0.304 |
| (μM) | 0.0001 | 0.596 | >10 | >10 | 0.525 | 0.084 |
| | 0.001 | 0.039 | 0.054 | 0.069 | 0.074 | 0.02 |
| | 0.1 | 0.916 | 0.897 | 0.962 | 0.563 | 0.076 |
| | 10 | >10 | >10 | >10 | 8.641 | 0.458 |

TABLE 6 (k)
BT474: Poziotinib + Trametinib

| | Trametinib (μM) | | | | | |
|---|---|---|---|---|---|---|
| CI value | | 0.100 | 0.32 | 1.00 | 3.16 | 10 |
| Poziotinib | 0.00001 | 0.0182 | 1.021 | 0.673 | 5.536 | 9.675 |
| (μM) | 0.0001 | 0.0692 | 0.144 | 0.391 | 4.301 | 2.741 |
| | 0.001 | 0.3805 | 0.018 | 0.041 | 0.088 | 0.188 |
| | 0.1 | 0.515 | 0.468 | 0.484 | 0.514 | 0.483 |
| | 10 | 0.678 | 18.315 | 17.478 | 16.432 | 9.474 |

TABLE 6 (l)
BT474: Poziotinib + Pemetrexed

| | Pemetrexed (μM) | | | | | |
|---|---|---|---|---|---|---|
| CI value | | 0.100 | 0.32 | 1.00 | 3.16 | 10 |
| Poziotinib | 0.00001 | 9.323 | 2.735 | 0.071 | 1.265 | 1.724 |
| (μM) | 0.0001 | 0.057 | 0.019 | 0.016 | 0.043 | 0.103 |
| | 0.001 | 0.011 | 0.011 | 0.011 | 0.013 | 0.015 |
| | 0.1 | 0.458 | 0.475 | 0.462 | 0.538 | 0.418 |
| | 10 | 12.802 | 11.884 | 12.781 | 13.95 | 10.914 |

TABLE 6 (m)
SK-BR-3: Poziotinib +
T-DM1

| | T-DM1(μg/ml) | | | | | |
|---|---|---|---|---|---|---|
| CI value | | 0.0002 | 0.0020 | 0.0200 | 0.2000 | 2 |
| Poziotinib | 0.0001 | 2.316 | 6.506 | 5.088 | 0.353 | 2.284 |
| (μM) | 0.0004 | 0.851 | 0.989 | 1.106 | 0.457 | 2.775 |
| | 0.0011 | 0.693 | 0.714 | 0.676 | 0.759 | 2.775 |
| | 0.0033 | 0.817 | 0.822 | 0.848 | 0.995 | 2.775 |
| | 0.01 | 1.679 | 1.682 | 1.662 | 1.752 | 3.004 |

This study was undertaken to further evaluate the possible synergy effect of the above combinations on Calu-3, BT-474 and SK-BR-3 cell lines. From the analysis using the combination index (CI), most combinations demonstrated synergy especially upon treatment of two test compounds in their respective middle concentration ranges.

4. Discussion

As shown in above Tables the combination of poziotinib and another drug, according to an embodiment, exhibited a synergy effect in a growth inhibition test with respect to cells of breast cancer, stomach cancer, lung cancer, or esophageal cancer, in which the HER1 or HER 2 gene was overexpressed or mutated.

In the case of non-small-cell lung carcinoma (NSCLC,) an excellent synergistic effect was exhibited upon administration of paclitaxel or cisplatin in combination with poziotinib in HCC827 cells having the exon19del mutation, an activating mutation for Her1. When poziotinib was combined with 5-FU, no synergistic effect was observed under the same test conditions. In H1975 cells, a cell line having resistance to first generation EGFR TKIs due to the presence of the T790M mutation, an excellent synergistic effect was exhibited when poziotinib was combined with cetuximab, which is an EGFR antibody, at a concentration equal to or lower than GI₅₀ of poziotinib alone. In the H1975 cells, when poziotinib was combined with paclitaxel or 5-FU, a synergistic effect was exhibited at some concentrations, but no synergy was observed when poziotinib was combined with cisplatin under the same test conditions.

In the case of breast cancer, a high synergistic effect was exhibited when poziotinib was combined with paclitaxel, cisplatin, or 5-FU in BT-474 cells, which are Her2-overexpressed and negative to an estrogen receptor (ER). When poziotinib was combined with trastuzumab, a weak synergistic effect was exhibited at some concentrations equal to or lower than GI₅₀ of poziotinib alone. When poziotinib was combined with vinorelbine, a synergistic effect was not observed at concentrations equal to or lower than G150 of poziotinib alone. Similarly, a sufficient effect was observed to indicate synergy when vinorelbine was combined with poziotinib in SK-BR-3 cells, which are Her2-overexpressed and ER-negative. However, the combination of poziotinib with vinorelbine exhibited a synergistic effect at some concentrations in MDA-MB-361 cells, which are HERr2-overexpressed, ER-positive, and a trastuzumab resistant cell line, and this combination exhibited synergy at every concentration in MCF-7 cells, which are not HER1 and HER2-overexpressed and ER-positive.

Also, a synergistic effect was observed by the combination of poziotinib and vinorelbine at some concentrations in MDA-MB-468 cells, which are HER2 negative, ER-negative, and HER1-overexpressed triple negative breast cancer cells. In MBA-MB-453 cells, which are cells in which HER2 is overexpressed among trastuzumab resistant breast cancer cells, an excellent synergistic effect was observed at concentrations equal to or lower than GI₅₀ of poziotinib alone when paclitaxel, 5-FU, cisplatin, or trastuzumab was combined with poziotinib.

The synergistic effect of combining poziotinib with other drugs was also excellent when poziotinib was combined with 5-FU in TE cells, which is a HER2-overexpressed esophageal cancer cell line. In addition, when poziotinib was combined with trastuzumab in N-87 cells, which is a HER2-overexpressed gastric cancer cell line, a synergistic effect was observed at some concentrations.

These results show that the combination of pozioitnib and other target anticancer agents or cytotoxic anticancer agents, according to an embodiment, is highly effective in cancers such as breast cancer, stomach cancer, lung cancer, and esophageal cancer, and can effectively inhibit cancer that is resistant to conventional therapeutic agents. It is confirmed that the combination according to an embodiment can be effective even for cancer cells that do not have HER1 or HER2 overexpression, or mutants thereof.

As described above, according to one or more embodiments, a combination drug is provided for treating a neoplasm associated with overexpression or amplification of at least one gene of HER1, HER2, and HER4, or a mutant of HER1, HER2, or HER4 in a subject, and the combination may be used to effectively treat a neoplasm.

According to another embodiment, a method is provided for treating a neoplasm associated with overexpression or amplification of at least one gene of HER1, HER2, and HER4, or a mutant of HER1, HER2, or HER4 in a subject; when this method is used in a patient in need thereof, a neoplasm may be effectively treated.

According to another embodiment, a combination kit and a pharmaceutical composition for treating a neoplasm associated with overexpression or amplification of at least one gene of HER1, HER2, and HER4, or a mutant of HER1, HER2, or HER4 in a subject may be used to effectively treat a neoplasm.

Other aspects of the present invention includes novel combinations and drug regimens of a combination of poziotinib with at least one cytotoxic agent and/or at least one molecularly targeted agent, as the active ingredient, wherein the at least one cytotoxic agent is selected from the group consisting of taxanes, base analogs, platinum-based antineoplastic drugs and vinca alkaloids; and wherein the at least one molecularly targeted agent is selected from the group consisting of at least one epidermal growth factor receptor (EGFR) family inhibitor. The EGFR family inhibitor can be an anti-EGFR family antibody. The combination can comprise poziotinib and an anti-EGFR family antibody, where the anti-EGFR family antibody can be trastuzumab, margetuximab cetuximab, matuzumab, panitumumab, necitumumab, or pertuzumab. The epidermal growth factor receptor (EGFR) family inhibitor can be a mTOR inhibitor. The combination can comprise poziotinib and a taxane. The taxane can be selected from the group consisting of paclitaxel, docetaxel and cabazitaxel. The combination can comprise poziotinib and a base analog. The base analog can be selected from the group consisting of 5-fluorouracil, 6-mercaptopurine, capecitabine, gemcitabine, pemetrexed, methotrexate, cladribine, cytarabine, doxifludine, floxuridine, fludarabine and decarbazine. The combination can comprise poziotinib and a platinum-based antineoplastic drug. The platinum-based antineoplastic drug can be selected from the group consisting of cisplatin, carboplatin, dicycloplatin, eptaplatin, lobaplatin, miriplatin, nedaplatin, oxaliplatin, picoplatin, and satraplatin. The combination can comprise poziotinib and a vinca alkaloid. The vinca alkaloid can be selected from the group consisting of vinblastine, vincristine, vinflunine, vinorelbine, vincaminol, vinburnine, vineridine and vindesine. The mTOR inhibitor can be selected from the group consisting of zotarolimus, umirolimus, temsirolimus, sirolimus, sirolimus NanoCrystal, sirolimus TransDerm, sirolimus-PNP, everolimus, biolimus A9, ridaforolimus, rapamycin, TCD-10023, DE-109, MS-R001, MS-R002, MS-R003, Perceiva, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, ABTL-0812, and pharmaceutically acceptable salts thereof or combinations thereof. The at least one cytotoxic agent can be selected from the group consisting of paclitaxel, cisplatin, 5-fluorouracil, vinorelbine and any combinations thereof. The at least one molecularly targeted agent can be selected from the group consisting of cetuximab, trastuzumab and any combinations thereof. The combination drug can be selected from the group consisting of: (A) poziotinib and paclitaxel; (B) poziotinib and cisplatin; (C) poziotinib and 5-fluorouracil; (D) poziotinib and cetuximab; (E) poziotinib and trastuzumab; and (F) poziotinib and vinorelbine.

For the combination drug regimen, the neoplasm can be selected from the group consisting of non-small cell lung cancer, breast cancer, gastric cancer, colon cancer, pancreatic cancer, prostate cancer, myeloma, head and neck cancer, ovarian cancer, esophageal cancer and metastatic cell carcinoma. The neoplasm can be selected from the group consisting of non-small cell lung cancer, breast cancer, gastric cancer and esophageal cancer. The neoplasm can be selected from the group consisting of: (i) non-small cell lung cancer carrying one or more EGFR mutations selected from L858R substitution, T790M substitution and/or deletion in exon 19, and/or one or more EGFR mutations selected from the group consisting of A763, A767, 5768, V769, D770, N771, P772, and H773 substitution and/or deletion in exon 20; (ii) estrogen receptor-negative breast cancer with overexpression of HER1 and/or HER2; (iii) estrogen receptor- and progesterone receptor-double positive breast cancer with HER2 being expressed but without overexpression thereof; (iv) trastuzumab-resistant breast cancer with overexpression of HER2; (v) HER1-overexpressing breast cancer triply negative with respect to HER2, progesterone receptor and estrogen receptor; (vi) esophageal cancer with overexpression of HER2; and (vii) gastric cancer with overexpression of HER2.

The combination drug and the neoplasm to be treated can be selected from the group consisting of: (1) poziotinib and paclitaxel for treating (a) non-small cell lung cancer carrying EGFR mutation of L858R substitution, T790M substitution and/or deletion in exon 19, and/or one or more EGFR mutations selected from the group consisting of A763, A767, 5768, V769, D770, N771, P772, and H773 substitution and/or deletion in exon 20, (b) estrogen receptor-negative breast cancer with overexpression of HER2 or (c) trastuzumab-resistant breast cancer with overexpression of HER2; (2) poziotinib and cisplatin for treating (a) non-small cell lung cancer carrying EGFR mutation of L858R substitution, T790M substitution and/or deletion in exon 19, and/or one or more EGFR mutations selected from the group consisting of A763, A767, S768, V769, D770, N771, P772, and H773 substitution and/or deletion in exon 20, (b) estrogen receptor-negative breast cancer with overexpression of HER2 or (c) trastuzumab-resistant breast cancer with overexpression of HER2; (3) poziotinib and 5-fluorouracil for treating (a) estrogen receptor-negative breast cancer with overexpression of HER2, (b) trastuzumab-resistant breast cancer with overexpression of HER2; or (c) esophageal cancer with overexpression of HER2; (4) poziotinib and cetuximab for treating non-small cell lung cancer carrying EGFR substitutions of L858R and T790M with overexpression of HER1 and/or one or more EGFR mutations selected from the group consisting of A763, A767, S768, V769, D770, N771, P772, and H773 substitution and/or deletion in exon 20; (5) poziotinib and trastuzumab for treating (a) trastuzumab-resistant breast cancer with overexpression of HER2 or (b) gastric cancer with overexpression of HER2; and (6) poziotinib and vinorelbine for treating (a) estrogen receptor-negative breast cancer with overexpression of HER2, (b) estrogen receptor- and progesterone receptor-double positive and trastuzumab-resistant breast cancer with overexpression of HER2, (c) estrogen receptor- and progesterone receptor-double positive breast cancer doubly negative with respect to overexpression of HER1 and HER2 or (d) HER1-overexpressing breast cancer triply negative with respect to HER2, estrogen receptor and progesterone receptor.

Another aspect of the invention is directed to a method of treating a neoplasm in a subject in need or at risk of developing a HER1, HER2, overexpressed cancer, wherein the method comprising administering to the subject, either separately or in combination, therapeutically effective amounts of poziotinib and at least one cytotoxic agent and/or at least one molecularly targeted agent, as the active ingredient, wherein the at least one cytotoxic agent is selected from the group consisting of taxanes, base analogs, platinum-based antineoplastic drugs and vinca alkaloids; and wherein the at least one molecularly targeted agent is selected from the group consisting of epidermal growth factor receptor (EGFR) family inhibitors and mammalian target of rapamycin (mTOR) inhibitors. The at least one cytotoxic agent of the method can be selected from the group consisting of paclitaxel, cisplatin, 5-fluorouracil, vinorelbine and any combinations thereof. The at least one molecularly targeted agent of the method can be selected from the group consisting of cetuximab, trastuzumab and any combinations thereof. The administration, either separately or in combination, can be selected from the group consisting of: (a) poziotinib and paclitaxel; (b) poziotinib and cisplatin; (c) poziotinib and 5-fluorouracil; (d) poziotinib and cetuximab; (e) poziotinib and trastuzumab; and (f) poziotinib and vinorelbine. The neoplasm of the method can be non-small cell lung cancer, breast cancer, stomach cancer, colon cancer, pancreatic cancer, prostate cancer, myeloma, head and neck cancer, ovarian cancer, esophageal cancer, or metastatic cell carcinoma. The neoplasm can be selected from the group consisting of non-small cell lung cancer, breast cancer, gastric cancer and esophageal cancer. The neoplasm can be selected from the group consisting of: (i) non-small cell lung cancer carrying one or more EGFR mutations selected from L858R substitution, T790M substitution and/or deletion in exon 19; and/or one or more EGFR mutations selected from the group consisting of A763, A767, S768, V769, D770, N771, P772, and H773 substitution and/or deletion in exon 20, (ii) estrogen receptor-negative breast cancer with overexpression of HER1 and/or HER2; (iii) estrogen receptor- and progesterone receptor-double positive breast cancer with HER2 being expressed but without overexpression thereof; (iv) trastuzumab-resistant breast cancer with overexpression of HER2; (v) HER1-overexpressing breast cancer triply negative with respect to HER1, HER2 and estrogen receptor; (vi) esophageal cancer with overexpression of HER2; and (vii) gastric cancer with overexpression of HER2.

The administration and the neoplasm to be treated of the method can be selected from the group consisting of: (1) poziotinib and paclitaxel for treating (a) non-small cell lung cancer carrying EGFR mutation of L858R substitution, T790M substitution and/or deletion in exon 19, and/or one or more EGFR mutations selected from the group consisting of A763, A767, 5768, V769, D770, N771, P772, and H773 substitution and/or deletion in exon 20, (b) estrogen receptor-negative breast cancer with overexpression of HER2, or (c) trastuzumab-resistant breast cancer with overexpression of HER2; (2) poziotinib and cisplatin for treating (a) non-small cell lung cancer carrying EGFR mutation of L858R substitution, T790M substitution and/or deletion in exon 19, and/or one or more EGFR mutations selected from the group consisting of A763, A767, 5768, V769, D770, N771, P772, and H773 substitution and/or deletion in exon 20, (b) estrogen receptor-negative breast cancer with overexpression of HER2, or (c) trastuzumab-resistant breast cancer with overexpression of HER2; (3) poziotinib and 5-fluorouracil for treating (a) estrogen receptor-negative breast cancer with overexpression of HER2, (b) trastuzumab-resistant breast cancer with overexpression of HER2, or (c) esophageal cancer with overexpression of HER2; (4) poziotinib and cetuximab for treating non-small cell lung cancer carrying EGFR substitutions of L858R and T790M with overexpression of HER1; and/or one or more EGFR mutations selected from the group consisting of A763, A767, S768, V769, D770, N771, P772, and H773 substitution and/or deletion in exon 20; (5) poziotinib and trastuzumab for treating (a) trastuzumab-resistant breast cancer with overexpression of HER2, or (b) gastric cancer with overexpression of HER2; and (6) poziotinib and vinorelbine for treating (a) estrogen receptor-negative breast cancer with overexpression of HER2, (b) estrogen receptor- and progesterone receptor-double positive and trastuzumab-resistant breast cancer with overexpression of HER2, (c) estrogen receptor- and progesterone receptor-double positive breast cancer doubly negative with respect to overexpression of HER1 and HER2, or (d) HER1-overexpressing breast cancer triply negative with respect to HER2, estrogen receptor and progesterone receptor.

Another aspect of the invention is directed to a kit for treating a neoplasm in a subject, the kit comprising a first part and a second part, wherein the first part comprises poziotinib, and the second part comprises at least one active ingredient selected from the group consisting of a cytotoxic agent and a molecularly targeted agent, and wherein the at least one cytotoxic agent is selected from the group consisting of taxanes, base analogs, platinum-based antineoplastic drugs and vinca alkaloids; and wherein the at least one molecularly targeted agent is selected from the group consisting of epidermal growth factor receptor (EGFR) family inhibitors and mammalian target of rapamycin (mTOR) inhibitors. The kit can further comprise a package insert comprising instructions for treating a neoplasm associated with overexpression or amplification of HER1, HER2, or HER4 or a mutant thereof in a subject.

It should be understood that embodiments described herein should be considered as illustrative only, without limiting the scope of the invention. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A combination drug for treating a neoplasm in a subject, the combination drug comprising a combination of poziotinib with at least one cytotoxic agent and/or at least one molecularly targeted agent, as the active ingredient,
wherein the at least one cytotoxic agent is selected from the group consisting of taxanes, base analogs, platinum-based antineoplastic drugs and vinca alkaloids; and
wherein the at least one molecularly targeted agent is selected from the group consisting of at least one epidermal growth factor receptor (EGFR) family inhibitor.

2. The combination drug of claim 1, wherein the EGFR family inhibitor is an anti-EGFR family antibody.

3. The combination drug of claim 1, wherein the combination comprises poziotinib and an anti-EGFR family antibody.

4. The combination drug of claim 3, wherein the anti-EGFR family antibody is trastuzumab, T-DM1 margetuximab cetuximab, matuzumab, panitumumab, necitumumab, or pertuzumab.

5. The combination drug of claim 1, wherein the epidermal growth factor receptor (EGFR) family inhibitor is a mTOR inhibitor.

6. The combination drug of claim 1, wherein the combination comprises poziotinib and a taxane.

7. The combination drug of claim 6, wherein the taxane is selected from the group consisting of paclitaxel, docetaxel and cabazitaxel.

8. The combination drug of claim 1, wherein the combination comprises poziotinib and a base analog.

9. The combination drug of claim 8, wherein the base analog is selected from the group consisting of 5-fluorouracil, 6-mercaptopurine, capecitabine, gemcitabine, pemetrexed, methotrexate, cladribine, cytarabine, doxifludine, floxuridine, fludarabine and decarbazine.

10. The combination drug of claim 1, wherein the combination comprises poziotinib and a platinum-based antineoplastic drug.

11. The combination drug of claim 10, wherein the platinum-based antineoplastic drug is selected from the group consisting of cisplatin, carboplatin, dicycloplatin, eptaplatin, lobaplatin, miriplatin, nedaplatin, oxaliplatin, picoplatin, and satraplatin.

12. The combination drug of claim 1, wherein the combination comprises poziotinib and a vinca alkaloid.

13. The combination drug of claim 12, wherein vinca alkaloid is selected from the group consisting of vinblastine, vincristine, vinflunine, vinorelbine, vincaminol, vinburnine, vineridine and vindesine.

14. The combination drug of claim 5, wherein the mTOR inhibitor is selected from the group consisting of zotarolimus, umirolimus, temsirolimus, sirolimus, sirolimus NanoCrystal, sirolimus TransDerm, sirolimus-PNP, everolimus, biolimus A9, ridaforolimus, rapamycin, TCD-10023, DE-109, MS-R001, MS-R002, MS-R003, Perceiva, XL-765, quinacrine, PKI-587, PF-04691502, GDC-0980, dactolisib, CC-223, PWT-33597, P-7170, LY-3023414, INK-128, GDC-0084, DS-7423, DS-3078, CC-115, CBLC-137, AZD-2014, X-480, X-414, EC-0371, VS-5584, PQR-401, PQR-316, PQR-311, PQR-309, PF-06465603, NV-128, nPT-MTOR, BC-210, WAY-600, WYE-354, WYE-687, LOR-220, HMPL-518, GNE-317, EC-0565, CC-214, ABTL-0812, and pharmaceutically acceptable salts thereof or combinations thereof.

15. The combination drug of claim 1, wherein the at least one cytotoxic agent is selected from the group consisting of paclitaxel, cisplatin, 5-fluorouracil, vinorelbine and any combinations thereof.

16. The combination drug of claim 1, wherein the at least one molecularly targeted agent is selected from the group consisting of cetuximab, trastuzumab, T-DM1 and any combinations thereof.

17. The combination drug of claim 1, wherein the combination is selected from the group consisting of:
(A) poziotinib and paclitaxel;
(B) poziotinib and cisplatin;
(C) poziotinib and 5-fluorouracil;
(D) poziotinib and cetuximab;
(E) poziotinib and trastuzumab;
(F) poziotinib and T-DM1; and
(G) poziotinib and vinorelbine.

18. The combination drug of claim 1, wherein the neoplasm is selected from the group consisting of non-small cell lung cancer, breast cancer, gastric cancer, colon cancer, pancreatic cancer, prostate cancer, myeloma, head and neck cancer, ovarian cancer, esophageal cancer and metastatic cell carcinoma.

19. The combination drug of claim 18, wherein the neoplasm is selected from the group consisting of non-small cell lung cancer, breast cancer, gastric cancer and esophageal cancer.

20. The combination drug of claim 19, wherein the neoplasm is selected from the group consisting of:
(i) non-small cell lung cancer carrying one or more EGFR mutations selected from L858R substitution, T790M substitution and/or deletion in exon 19, and/or one or more EGFR mutations selected from the group consisting of A763, A767, S768, V769, D770, N771, P772, and H773 substitution and/or deletion in exon 20;
(ii) estrogen receptor-negative breast cancer with overexpression of HER1 and/or HER2;
(iii) estrogen receptor- and progesterone receptor-double positive breast cancer with HER2 being expressed but without overexpression thereof;
(iv) trastuzumab-resistant breast cancer with overexpression of HER2;
(v) HER1-overexpressing breast cancer triply negative with respect to HER2, progesterone receptor and estrogen receptor;
(vi) esophageal cancer with overexpression of HER2; and
(vii) gastric cancer with overexpression of HER2.

21. The combination drug of claim 1, wherein the combination and the neoplasm to be treated are selected from the group consisting of:
(1) poziotinib and paclitaxel for treating
(a) non-small cell lung cancer carrying EGFR mutation of L858R substitution, T790M substitution and/or deletion in exon 19, and/or one or more EGFR mutations selected from the group consisting of A763, A767, S768, V769, D770, N771, P772, and H773 substitution and/or deletion in exon 20,
(b) estrogen receptor-negative breast cancer with overexpression of HER2 or
(c) trastuzumab-resistant breast cancer with overexpression of HER2;
(2) poziotinib and cisplatin for treating
(a) non-small cell lung cancer carrying EGFR mutation of L858R substitution, T790M substitution and/or deletion in exon 19, and/or one or more EGFR mutations selected from the group consisting of A763, A767, S768, V769, D770, N771, P772, and H773 substitution and/or deletion in exon 20,
(b) estrogen receptor-negative breast cancer with overexpression of HER2 or
(c) trastuzumab-resistant breast cancer with overexpression of HER2;
(3) poziotinib and 5-fluorouracil for treating
(a) estrogen receptor-negative breast cancer with overexpression of HER2,
(b) trastuzumab-resistant breast cancer with overexpression of HER2; or
(c) esophageal cancer with overexpression of HER2;
(4) poziotinib and cetuximab for treating non-small cell lung cancer carrying EGFR substitutions of L858R and T790M with overexpression of HER1 and/or one or more EGFR mutations selected from the group consisting of A763, A767, 5768, V769, D770, N771, P772, and H773 substitution and/or deletion in exon 20;
(5) poziotinib and trastuzumab for treating
(a) trastuzumab-resistant breast cancer with overexpression of HER2 or
(b) gastric cancer with overexpression of HER2; and
(6) poziotinib and vinorelbine for treating
(a) estrogen receptor-negative breast cancer with overexpression of HER2,
(b) estrogen receptor- and progesterone receptor-double positive and trastuzumab-resistant breast cancer with overexpression of HER2,
(c) estrogen receptor- and progesterone receptor-double positive breast cancer doubly negative with respect to overexpression of HER1 and HER2 or
(d) HER1-overexpressing breast cancer triply negative with respect to HER2, estrogen receptor and progesterone receptor.

22. A method of treating a neoplasm in a subject, the method comprising administering to the subject, either separately or in combination, therapeutically effective amounts of poziotinib and at least one cytotoxic agent and/or at least one molecularly targeted agent, as the active ingredient,
wherein the at least one cytotoxic agent is selected from the group consisting of taxanes, base analogs, platinum-based antineoplastic drugs and vinca alkaloids; and
wherein the at least one molecularly targeted agent is selected from the group consisting of epidermal growth factor receptor (EGFR) family inhibitors and mammalian target of rapamycin (mTOR) inhibitors.

23. The method of claim 22, wherein the at least one cytotoxic agent is selected from the group consisting of paclitaxel, cisplatin, 5-fluorouracil, vinorelbine and any combinations thereof.

24. The method of 22, wherein the at least one molecularly targeted agent is selected from the group consisting of cetuximab, trastuzumab, T-DM1 and any combinations thereof.

25. The method of claim 22, wherein the administration is selected from the group consisting of:
(a) poziotinib and paclitaxel;
(b) poziotinib and cisplatin;
(c) poziotinib and 5-fluorouracil;
(d) poziotinib and cetuximab;
(e) poziotinib and trastuzumab;
(f) poziotinib and T-DM1; and
(g) poziotinib and vinorelbine.

26. The method of claim 22, wherein the neoplasm is non-small cell lung cancer, breast cancer, stomach cancer, colon cancer, pancreatic cancer, prostate cancer, myeloma, head and neck cancer, ovarian cancer, esophageal cancer, or metastatic cell carcinoma.

27. The method of claim 26, wherein the neoplasm is selected from the group consisting of non-small cell lung cancer, breast cancer, gastric cancer and esophageal cancer.

28. The method of claim 27, wherein the neoplasm is selected from the group consisting of:
(i) non-small cell lung cancer carrying one or more EGFR mutations selected from L858R substitution, T790M substitution and/or deletion in exon 19; and/or one or more EGFR mutations selected from the group consisting of A763, A767, 5768, V769, D770, N771, P772, and H773 substitution and/or deletion in exon 20,
(ii) estrogen receptor-negative breast cancer with overexpression of HER1 and/or HER2;
(iii) estrogen receptor- and progesterone receptor-double positive breast cancer with HER2 being expressed but without overexpression thereof;
(iv) trastuzumab-resistant breast cancer with overexpression of HER2;
(v) HER1-overexpressing breast cancer triply negative with respect to HER1, HER2 and estrogen receptor;
(vi) esophageal cancer with overexpression of HER2; and
(vii) gastric cancer with overexpression of HER2.

29. The method of claim 22, the administration and the neoplasm to be treated are selected from the group consisting of:
(1) poziotinib and paclitaxel for treating
(a) non-small cell lung cancer carrying EGFR mutation of L858R substitution, 1790M substitution and/or deletion in exon 19, and/or one or more EGFR mutations selected from the group consisting of A763, A767, 5768, V769, D770, N771, P772, and H773 substitution and/or deletion in exon 20,
(b) estrogen receptor-negative breast cancer with overexpression of HER2, or
(c) trastuzumab-resistant breast cancer with overexpression of HER2;
(2) poziotinib and cisplatin for treating
(a) non-small cell lung cancer carrying EGFR mutation of L858R substitution, T790M substitution and/or deletion in exon 19, and/or one or more EGFR mutations selected from the group consisting of A763, A767, S768, V769, D770, N771, P772, and H773 substitution and/or deletion in exon 20,
(b) estrogen receptor-negative breast cancer with overexpression of HER2, or
(c) trastuzumab-resistant breast cancer with overexpression of HER2;
(3) poziotinib and 5-fluorouracil for treating
(a) estrogen receptor-negative breast cancer with overexpression of HER2,
(b) trastuzumab-resistant breast cancer with overexpression of HER2, or
(c) esophageal cancer with overexpression of HER2;
(4) poziotinib and cetuximab for treating non-small cell lung cancer carrying EGFR substitutions of L858R and T790M with overexpression of HER1; and/or one or more EGFR mutations selected from the group consisting of A763, A767, S768, V769, D770, N771, P772, and H773 substitution and/or deletion in exon 20,
(5) poziotinib and trastuzumab for treating
(a) trastuzumab-resistant breast cancer with overexpression of HER2, or
(b) gastric cancer with overexpression of HER2; and
(6) poziotinib and vinorelbine for treating
(a) estrogen receptor-negative breast cancer with overexpression of HER2,
(b) estrogen receptor- and progesterone receptor-double positive and trastuzumab-resistant breast cancer with overexpression of HER2,
(c) estrogen receptor- and progesterone receptor-double positive breast cancer doubly negative with respect to overexpression of HER1 and HER2, or
(d) HER1-overexpressing breast cancer triply negative with respect to HER2, estrogen receptor and progesterone receptor.

30. A kit for treating a neoplasm in a subject, the kit comprising a first part and a second part, wherein the first part comprises poziotinib, and the second part comprises at least one active ingredient selected from the group consisting of a cytotoxic agent and a molecularly targeted agent, and
wherein the at least one cytotoxic agent is selected from the group consisting of taxanes, base analogs, platinum-based antineoplastic drugs and vinca alkaloids; and
wherein the at least one molecularly targeted agent is selected from the group consisting of epidermal growth factor receptor (EGFR) family inhibitors and mammalian target of rapamycin (mTOR) inhibitors.

31. The kit of claim 30, further comprising a package insert comprising instructions for treating a neoplasm associated with overexpression or amplification of HER1, HER2, or HER4 or a mutant thereof in a subject.
